SEQUENCE LISTING (8 sequences)

SEQ ID NO: 1 - DNA, 21 bp, Homo sapiens
  misc_feature 1..21: note = beta-actin forward primer
  source 1..21: mol_type = unassigned DNA
catgtacgttgctatccaggc

SEQ ID NO: 2 - DNA, 21 bp, Homo sapiens
  misc_feature 1..21: note = beta-actin reverse primer
  source 1..21: mol_type = unassigned DNA
ctccttaatgtcacccacgat

SEQ ID NO: 3 - DNA, 20 bp, Homo sapiens
  misc_feature 1..20: note = TNF-alpha forward primer
  source 1..20: mol_type = unassigned DNA
atccgagatgtggaactggc

SEQ ID NO: 4 - DNA, 22 bp, Homo sapiens
  misc_feature 1..22: note = TNF-alpha reverse primer
  source 1..22: mol_type = unassigned DNA
tgggaacttctcctccttgttg

SEQ ID NO: 5 - DNA, 20 bp, Homo sapiens
  misc_feature 1..20: note = IL-6 forward primer
  source 1..20: mol_type = unassigned DNA
agtgaggaacaagccagagc

SEQ ID NO: 6 - DNA, 21 bp, Homo sapiens
  misc_feature 1..21: note = MCP-1 forward primer
  source 1..21: mol_type = unassigned DNA
catagcagccaccttcattcc

SEQ ID NO: 7 - DNA, 20 bp, Homo sapiens
  misc_feature 1..20: note = MCP-1 reverse primer
  source 1..20: mol_type = unassigned DNA
catagcagccaccttcattc

SEQ ID NO: 8 - DNA, 20 bp, Homo sapiens
  misc_feature 1..20: note = MCP-1 reverse primer
  source 1..20: mol_type = unassigned DNA
tctccttggccacaatggtc

(19) United States
(12) Patent Application Publication
(10) Pub. No.: US 2026/0060947 A1
(43) Pub. Date: Mar. 5, 2026

(54) ELECTROPHILIC NITROALKENE BENZOIC ACID DERIVATES AS THERAPEUTIC DRUGS IN AMYOTROPHIC LATERAL SCLEROSIS (ALS) AND OTHER NEURODEGENERATIVE CONDITIONS

(71) Applicants: INSTITUT PASTEUR DE MONTEVIDEO, Montevideo (UY); UNIVERSIDAD DE LA REPÚBLICA, Montevideo (UY)

(72) Inventors: Luis Barbeito, Montevideo (UY); Emiliano Trías, Montevideo (UY); Sofía Ibarburu, Montevideo (UY); Carlos Batthyány, Montevideo (UY); Carlos Escande, Montevideo (UY); Gloria Virginia López, Montevideo (UY); Williams Arturo Porcal Quinta, Montevideo (UY); Mariana Ingold, Montevideo (UY); Lucia Colella, Montevideo (UY)

(73) Assignees: INSTITUT PASTEUR DE MONTEVIDEO, Montevideo (UY); UNIVERSIDAD DE LA REPÚBLICA, Montevideo (UY)

(21) Appl. No.: 19/093,370

(22) Filed: Mar. 28, 2025

Related U.S. Application Data
(63) Continuation of application No. 18/526,028, filed on Dec. 1, 2023, now abandoned.
Application No. 18/526,028 is a continuation of application No. 17/080,523, filed on Oct. 26, 2020, now abandoned.
(60) Provisional application No. 62/925,383, filed on Oct. 24, 2019.

(51) Int. Cl. A61K 31/192; A61P 25/28

(52) U.S. Cl. CPC A61K 31/192; A61P 25/28

(57) ABSTRACT

This invention relates to the use of nitroalkene derivatives for the treatment of neurodegenerative conditions in mammals in which neuroinflammation is a contributing factor, such as in amyotrophic lateral sclerosis (ALS).

### CROSS REFERENCE TO RELATED APPLICATIONS

This application is a continuation of U.S. Non-provisional application Ser. No. 18/526,028, filed on 1 Dec. 2023, which is a continuation of U.S. Non-provisional application Ser. No. 17/080,523, filed on 26 Oct. 2020 and claims the benefit of the filing date of U.S. Provisional Application No. 62/925,383, filed on 24 Oct. 2019, the contents of which are incorporated by reference herein in the entirety.

### SEQUENCE LISTING

The instant application contains a Sequence Listing which has been submitted electronically in XML file format and is hereby incorporated by reference in its entirety. Said XML copy, created on Feb. 14, 2024, is named IPDM8US-CON_00598103_SL.xml and is 11,270 bytes in size.

### FIELD OF INVENTION

This invention relates to nitroalkene derivatives for the treatment of amyotrophic lateral sclerosis (ALS) and related neurodegenerative conditions where neuroinflammation contributes to neuronal degeneration and to the ineluctable progression of neurological deficits that characterize neurodegenerative conditions. Current drugs for these indications are not curative and only have a modest effect in disease progression or survival.

### BACKGROUND

There is no curative treatment for amyotrophic lateral sclerosis (ALS), a paralytic disease characterized by the gradual degeneration of motor neurons that control muscles. Survival after diagnosis (3-5 years) is largely determined by the rate of spread of motor neuron pathology along the neuroaxis [1]. ALS etiology remains largely unknown and there is a poor understanding of the pathological mechanisms underlying the disease onset and subsequent progressive spreading. Currently approved drugs for ALS, riluzole and edaravone, have only a modest and clinically irrelevant therapeutic effects in most patients [2, 3]. Clinical trials have shown that riluzole extends survival by a few months, while edaravone improves the daily functioning in a restricted subset of ALS subjects. Thus, there is an unmet need to develop new treatments to slow or stop the paralysis progression early after diagnosis, with the hope of turning this fatal disease into a chronic condition.

There is evidence that paralysis progression in rodent models of ALS is modulated by glial cells that proliferate and express inflammatory mediators in the degenerating spinal cord [4-7]. In particular, the proliferation and accumulation of microglial cells (microgliosis) and the subsequent emergence of aberrant glial cells are major neuropathological features for ALS animal models [7-9]. In ALS patients, microglia activation can be observed in the motor cortex, corticospinal tract and ventral horn of the spinal cord [10]. Activated microglia contribute to oxidative stress and the local and systemic production of inflammatory cytokines, with evidence of their upregulation found in ALS patients as well as animal models [11]. NF-κB signaling in microglia is causally associated with their neurotoxic potential to motor neurons [12]. Pharmacological inhibition of dysfunctional reactive microglia may prolong survival in rodent ALS models or prevent glia-induced motor neuron death in culture conditions [13, 14].

In the SOD1^{G93A} mutant rat model of ALS, a rapid spread of paralysis is associated with marked glial cell activation and the emergence of aberrant glial cells that actively proliferate around degenerating motor neurons [4, 5]. Furthermore, aberrant glial cells display a marked neurotoxic potential on cultured motor neurons [4], suggesting that they might directly contribute to the rapid spread of paralysis of ALS rats. Downregulation of microglia and aberrant glial cells using tyrosine kinase inhibitors have been shown to slow paralysis progression in SOD1^{G93A} rats, even when treatment starts up to seven (7) days after disease onset [13].

The transcription nuclear factor κ light-chain-enhancer of activated B cells (NF-κB) is widely distributed among various cell types in the CNS being involved in many physiological and pathological processes [10,11]. NF-κB is constitutively expressed as a dimer usually formed by subunits p50 and p65, which dissociate after activation to enter the cell nucleus and promote the transcription of target genes involved in cell survival, proliferation, and inflammation [32]. In ALS models, activation of the transcriptional factor NF-κB in non-neuronal cells including microglia plays a crucial pathogenic role [12]. While NF-κB-mediated transcription occurs physiologically in neurons and astrocytes [33], NF-κB activation and subsequent transcriptional activity in microglia appear as a distinctive feature of ALS and other neurodegenerative conditions such as Alzheimer's disease [34]. In the ALS SOD1^{G93A} mouse model, microglia-mediated motor neuron death occurs through an NF-κB-dependent mechanism and constitutive activation of NF-κB in microglia causes accelerated loss of motor neurons through a non-cell-autonomous mechanism [12]. Additionally, NF-κB is highly induced in microglia of sporadic ALS patients [35] and those with a mutation in optineurin, a negative regulator of TNFα which induces NF-κB activation [36]. In transgenic mice overexpressing mutant TDP-43, the protein acts as an NF-κB coactivator, and NF-κB inhibition was shown to be protective [35]. Therefore, aberrant NF-κB activation in ALS appears to endow microglia with a neurotoxic phenotype, which could be potentially pharmacologically targeted. However, there is scarce knowledge about NF-κB inhibitor drugs targeting neurotoxic microglia in ALS.

The recently FDA-approved dimethyl fumarate can downregulate neuroinflammation and immune response in multiple sclerosis and also in ALS experimental models [15-17], by inducing Nrf2/keap1 and inhibiting NF-κB-transcriptional pathways [18].

Nitrated unsaturated fatty acids, considered as endogenous nitroalkenes with electrophilic properties, also have the ability to modulate Nrf2/keap1 [19, 20] and NF-κB-pathways [21, 22] in a variety of target cells including astrocytes, improving motor deficits and reducing neuroinflammation in an ALS mouse model [23]. The anti-inflammatory effects of electrophilic fatty acid nitroalkene derivatives are also associated with activation of PPAR-γ [24] and the Heat Shock Response [25].

Summarily, current approved drugs for ALS have only a modest and clinically irrelevant therapeutic effects in most patients. Thus, there is an unmet need to develop new treatments to slow or stop the paralysis progression early after diagnosis.

### BRIEF DESCRIPTION OF THE FIGURES AND DRAWINGS

FIG. 1A: Synthesis of (E)-4-(2-nitrovinyl) benzoic acid (BANA).

FIG. 1B: Spectrophotometric characterization of the reaction between BANA (30 μM) and β-Mercaptoethanol (300 μM). UV-Visible spectra were analyzed. Scans were taken each min up to 15 min.

FIG. 1C: Plasma levels of BANA as measured by HPLC. Upper panels show calibration curve and chromatogram of BANA and S.A. and BANA concentrations in plasma after 15 days of treatment (table at right). Plasma chromatograms (n=2 control; n=4 BANA) are shown in the graphs below.

FIG. 2A: For inflammasome activation, THP-1 cells were treated with LPS and ATP. Graphs showing the concentration of IL1-1α in THP-1 cell's supernatant when cells were treated with benzoic acid (B.A, 30 μM) or BANA (30 μM) during the first signal (LPS, left) or in second signal (ATP, right). All data are expressed as mean±SD; *p<0.05.

FIG. 2B: Graphs showing the LPS-induced gene expression in THP-1 cells treated with vehicle, BANA (30 μM) or benzoic acid (B.A, 30 μM). Note the increased in NF-κB-dependent transcripts for TNFα, MCP-1 and IL-6 were abrogated by treatment with BANA but not by benzoic acid. Data are expressed as mean±SD; *p<0.05.

FIG. 3A: IC50 graph of murine BV2 microglial cell line, treated with increasing concentrations of BANA (5 μM-110 μM). The IC50 value for BANA was 51.7 μM.

FIG. 3B: For inflammasome activation, BV2 cells were treated with LPS and ATP. BANA or B.A treatment was performed in second signal (ATP). Presence of pro-IL1β in cell lysates (CL) and cleaved IL-1β in supernatants (SN) was determined by western blot analysis. The graph below shows cleaved IL-1β/pro-IL1-1β ratio. All data are expressed as mean±SEM.

FIG. 3C: Confocal images of BV2 microglial cells treated with BANA before LPS stimulation (left). White arrows show p65-NFkB positive nuclei (Scale bar=10 m). Note that those cells pre-treated with BANA have reduce p65-NFkB positive nucleus (graph at right).

FIG. 4A: Scheme of the experimental design. Rats were treated with BANA or vehicle at paralysis onset until final stages of the disease.

FIG. 4B: Kaplan-Meier survival curves from BANA and vehicle-treated SOD1^{G93A} rats. SOD1^{G93A} transgenic rats were treated with BANA 100 mg/kg/day (n=10 green line) or vehicle (PB, n=10 black line) immediately after paralysis onset of one hindlimb. There was a statistically significant difference in the probability of survival for BANA-treated group when compared with vehicle-treated group, according to the log-rank test of the Kaplan-Meier analysis (p=0.0007).

FIG. 4C: A graph showing the mean survival of each group. There was a statically significant difference between groups according to Dunn's multiple comparisons test (p=0.0018). All data are expressed as mean±SEM.

FIG. 5A: Representative confocal microphotographs showing the microglia markers Iba1-(upper panels), CD68- (middle panels) and CD34- (lower panels) positive cells in the ventral horn of lumbar spinal cord. Non-transgenic, and SOD1^{G93A} rats at onset and 15 d after onset (vehicle and BANA) were analyzed (Scale bar=50 μm for Iba1 and CD34 and 10 μm for CD68). Graphs at the right show the quantification of mean fluorescence intensity of Iba1, CD68 and CD34, which correlates quantitatively to microgliosis in the ventral spinal cord. Dunn's multiple comparisons test was performed between groups for each marker. All data are shown as mean±SEM; **P=0.0059/***p=0.0002.

FIG. 5B: Confocal images showing Ki67+ proliferating among groups (Scale bar=50 m). Graphs at the right show the number of Ki67+ nuclei/area. Dunn's multiple comparisons test was performed between group. Data is shown as mean±SEM; **p=0.0065.

FIG. 6: Upper panels show low magnification confocal images showing GFAP+ astrocytes measured in the ventral horn of lumbar spinal cord of Non-transgenic (Non-Tg), symptomatic onset, symptomatic vehicle or symptomatic BANA. Dotted lines separate gray from white matter. Lower panels show at higher magnification aberrant glial cells that surround motor neuron as estimated by co-staining with GFAP/S100f. Scale bar=50 nm and 10 um, respectively. The graphs at the right show GFAP mean fluorescence intensity for each condition (upper graph) and number of aberrant glial cell that surround motor neuron (lower graph). Note that BANA significantly reduced astrogliosis and aberrant glial cells when compare to vehicle. Dunn's multiple comparisons test was performed between group. All data are shown as mean±SEM; ****P<0.0001.

FIG. 7A: Representative confocal image of ChAT+ motor neurons in the ventral horn of lumbar spinal cord in non-transgenic, and SOD1^{G93A} rats at paralysis onset and 15 d after paralysis. Upper panels show representative low magnification images from the ventral spinal cord (dotted lines separate gray from white matter). Lower panels show motor neuron at higher magnification. Scale bar 50 μm in low magnification and 20 μm in high magnification. Graphs at the right show the quantitative analysis of the number and size of motor neurons in the ventral horn of lumbar spinal cord in each condition. Dunn's multiple comparisons test was performed between group. All data are shown as mean±SEM; *P=0.027/****P<0.0001. Note BANA treatment preserved motor neuron size and number as compared with vehicle.

FIG. 7B: Representative confocal images showing ubiquitin+ motor neuron. Scale bar=10 μm). Note decreased ubiquitin staining in animal treated with BANA respect to vehicle.

FIG. 8A: Data showing BANA inhibition of NF-κB activation in LPS challenge in vivo in NF-κB-RE-Luc reporter mouse. BANA at 20 and 30 mg/kg when administered 2 h before LPS intraperitoneal injection significantly inhibited NF-κB activation.

FIG. 8A-1: Scheme of the experimental design for the data displayed in FIG. 8A. BANA (50 mg/kg), BA (50 mg/kg) or vehicle, were intraperitoneally (IP) injected 1 h before LPS intraperitoneal administration (10 mg/kg). 3 h later, IL-1β levels were measured in the peritoneum and plasma by ELISA.

FIG. 8A-2: The bar graphs shows that BANA significantly reduces IL-1□ concentration in the peritoneum (left graph) and plasma (right graph) when compared to vehicle and BA. All data are expressed as mean±SEM; data were analyzed by Ordinary one-way ANOVA followed by Tukey's multiple comparisons test, ***p=0.0002 ****p<0,0001. n=3 animals per condition.

FIG. 8B: BANA inhibits NF-κB activation in NF-κB-RE-Luc transgenic mice. BANA, Benzoic acid (BA), or dimethyl fumarate (DMF) were administered 2 h before intraperitoneal injection of LPS. The graph shows that quantitative analysis under varying experimental conditions.

FIG. 8C: Confocal images of BV2 microglia treated with BANA, BA, or DMF before LPS stimulation. BANA inhibits LPS-induced NF-κB-p65 nuclear translocation. Cells in the sham condition were not treated with LPS. Nuclear NF-κB-p65 colocalizing with DAPI nuclear staining is denoted in yellow. The graph shows the quantitative analysis of the ratio nuclear NF-κB-p65/DAPI among experimental conditions.

FIG. 8D: Confocal images show CD11b-positive SOD1^{G93A} primary microglia (grey) treated with BANA, BA or vehicle, before LPS stimulation. BANA inhibits LPS-induced NF-κB-p65 nuclear translocation in SOD1^{G93A} microglia. Nuclear NF-κB-p65 colocalizing with DAPI nuclear staining is denoted in yellow.

FIG. 8E: A graph showing the quantitative analysis of the ratio nuclear NF-κB-p65/DAPI among various experimental conditions summarized in FIG. 8C.

FIG. 8F: Analysis by RT-qPCR of mRNA levels of NF-κB-associated proinflammatory cytokines CCL2, IL-6, IL-1β, and TNFα following LPS-stimulation in SOD1^{G93A} primary microglia. The data shows that BANA prevents NF-κB-mediated gene transcription induced by LPS.

FIG. 8G: A graph showing the quantitative analysis of NF-κB activation in different experimental conditions in which cells were exposed to BANA (10, 20 and 30 μM), BA, (30 μM) or DMF (30 μM) before stimulation with TNFα and then analyzed by flow cytometry. All quantitative data are expressed as mean±SEM; data were analyzed by Ordinary one-way ANOVA followed by Tukey's multiple comparisons, **p=0.0086 ****p<0,0001. 3 replicates of 4 independent experiments

FIG. 9A: Scheme of the experimental design. SOD1^{G93A} female rats were treated with 100 mg/kg/day of BANA or vehicle immediately after the first signs of paralysis onset of one hindlimb and continue for 15 days. Immunohistological analysis of the lumbar spinal cord of non-transgenic and vehicle- and BANA-treated SOD1^{G93A} symptomatic rats were performed.

FIG. 9B: Confocal images showing Iba1-positive microglia (white) in the ventral spinal cord. Yellow dotted lines delimit white from gray matter. Insets show microglia-associated motor neurons (MTN, yellow dotted lines).

FIG. 9C: Confocal images show NF-κB-p65 (green) and DAPI (red) expression in the ventral horn of the spinal cord of non-transgenic and symptomatic SOD1^{G93A} rats, treated with vehicle or BANA, in the surroundings of motor neurons (white dotted lines). Nuclei stained in yellow denote nuclear NF-κB-p65 and DAPI colocalization (white arrows). The graph to the right shows the quantitative analysis of the ratio NF-κB-p65/DAPI in the surroundings of motor neurons. Data are expressed as mean±SEM; data were analyzed by the Kruskal-Wallis test followed by Dunn's multiple comparisons test, **p=0.0018, ***p=0.0003. n=4 animals per condition. Scale bar=20 μm.

FIG. 9D: High magnification confocal images showing NF-κB-p65 nuclear expression in Isolectin-positive microglia (white) surrounding degenerating motor neurons stained with Nissl (blue) in the symptomatic SOD1^{G93A} ventral spinal cord. Scale bar=10 μm.

FIG. 10A: Scheme of an experimental design, the results of which are shown in FIGS. 10B-10C.

FIG. 10B: Representative confocal images showing the expression of nuclear NF-κB-p65 in the cellular microenvironment surrounding spinal motor neurons (white dotted lines) of ALS and control subjects. The “upper” panels show NF-κB-p65 staining (green) and DAPI (red) in control subjects. The right “upper” panel represents a higher magnification image of few Iba1-positive microglia (white) that lack cytoplasmatic or nuclear expression of NF-κB-p65 (green). The “lower” panels represent confocal images showing a systematic increase expression of nuclear NF-κB-p65 in the surroundings of motor neurons in 3 sporadic ALS subjects. Arrows indicate yellow nuclei with NF-κB-p65 colocalizing with DAPI. The graph to the lower right shows the quantitative analysis of NF-κB-p65-positive nuclei surrounding motor neurons. A 11 quantitative data are expressed as mean±SEM; data were analyzed by the Mann-Whitney test, ****p<0,0001. n=3 ALS patients and 3 controls. Scale bars=20 m,

FIG. 10C: Higher magnification confocal images showing the cytoplasmic and nuclear expression (magenta arrows) of NF-κB-p65 in Iba1-positive microglia in the ventral spinal cord of ALS subjects. Scale bars=10 μm.

FIG. 11A: Scheme of the experimental design, the results of which are shown in FIGS. 11B and 11C. SOD1^{G93A} female rats were treated with 100 mg/kg/day of BANA or vehicle immediately after paralysis onset until endstage (n=10 per group).

FIG. 11B: Kaplan-Meier survival curves from BANA- and vehicle-treated rats. There was a statistically significant difference in the probability of survival for the BANA-treated group when compared with the vehicle, according to the Log-rank test of the Kaplan-Meier analysis (***p=0,0007).

FIG. 11C: A graph showing the mean survival of both cohorts (i.e., BANA- and vehicle-treated rats). All data are expressed as mean±SEM; data were analyzed by Unpaired t-test **p=0.0018.

FIG. 12A: The “upper” panels show confocal images of ChAT-positive motor neurons in the lumbar spinal cord (dotted line indicates the limit between white and grey matter) of non-transgenic, vehicle- and BANA-treated SOD1^{G93A} rats immediately after the first signs of paralysis onset during 15 days. The upper graph represents the quantitative analysis of the number of motor neurons in laminae VII and IX of ventral horn among conditions. All data are expressed as mean±SEM; data were analyzed by the Kruskal-Wallis test followed by Dunn's multiple comparisons test, *p=0.0217 ****p<0.0001. n=4 animals per condition. Scale bar=50 m. The “lower” panels represent the immunohistochemical analysis of motor neuron soma size. The “lower” graph represents the quantitation of the motor neuron soma diameter in the different experimental conditions. All data are expressed as mean±SEM; data were analyzed by the Kruskal-Wallis test followed by Dunn's multiple comparisons test, ****p<0.0001. n=4 animals per condition. Scale bar=20 μm.

FIG. 12B: Immunohistochemical analysis of ubiquitin and nitrotyrosine (NO₂-Tyrosine) aggregation in motor neurons (yellow dotted lines) of the lumbar spinal cord. Vehicle-treated rats showed a significantly increased number of ubiquitinated motor neurons and increased expression of NO2Tyrosine when compared with non-transgenic littermates. Post-paralysis treatment with BANA significantly prevented ubiquitin aggregation and NO₂-Tyrosine accumulation in surviving motor neurons. The graph to the right shows the number of motor neurons showing ubiquitin and NO₂-Tyrosine aggregates in the ventral horn of the spinal cord. Data are expressed as mean±SEM; data were analyzed by the Kruskal-Wallis test followed by Dunn's multiple comparisons test, *p=0.0219 ****p<0.0001. n=4 animals per condition. Scale bars=20 μm.

FIG. 13A: Representative confocal images showing 0111-Tubulin-positive neurons (blue) in the ventral horn of lumbar spinal cord co-stained with synaptic vesicle markers VGlut-1 (green) and synaptophysin (red). Note that post-paralysis treatment with BANA after 15 d of from onset significantly prevented synaptic terminal loss that contact motor neuron cell bodies. Scale bar=10 μm.

FIG. 13B: NMJs denervation analysis in whole mounted EDL muscles. The panels show representative confocal images used to assess the innervation pattern of NMJs in different experimental conditions. a-bungarotoxin-FITC (red) staining was used to analyze motor endplates. Synaptophysin-AlexFluor 555 and heavy chain of neurofilaments-AlexaFluor 555 (green) were used to visualize the motor axon branches and presynaptic terminals. Arrows indicate typical denervated motor endplates. Scale bar=50 μm.

FIG. 13C: A graph showing the quantitative analysis of synaptic vesicles in contact with motor neuron cell bodies (left and middle graphs), and NMJ occupancy defined as the overlapping of neurofilament/synaptophysin and QBTX staining (graph to the right), expressed as percent respect the non-transgenic condition. All data are expressed as mean±SEM; data were analyzed by Mann-Whitney comparisons test or unpaired t-test between vehicle and BANA groups, *p=0.0474 (VGlut-1), *p=0.0447 (synaptophysin) *p=0.0324 (NMJ). n=4 animals per condition.

FIG. 14: Representative confocal images showing immunohistochemistry analysis of cell proliferation by nuclear Ki67 staining (green) in the surroundings of motor neurons (dotted white lines) in SOD1^{G93A} female rats that had been treated with 100 mg/kg/day of BANA or vehicle immediately after the first signs of paralysis onset of one hindlimb, and continuing for 15 days. The graph shows the quantitative analysis of the ratio Ki67/DAPI (green/red) in each condition. BANA significantly reduced cell proliferation when compared with the vehicle-treated group. All quantitative data are expressed as mean±SEM; data were analyzed by Ordinary one-way ANOVA followed by Tukey's multiple comparisons test, **p=0.0013 ****p<0,0001. n=4 animals per condition. Scale bars=15 μm.

FIG. 15A: Experimental design showing that SOD1^{G93A} female rats were treated with 50 mg/kg/day of BANA or vehicle immediately after the first signs of paralysis onset of one hind limb and continue for 15 days. Immunohistological analysis of the lumbar spinal cord of non-transgenic and vehicle- and BANA-treated SOD1^{G93A} symptomatic rats were then performed.

FIG. 15B: Representative confocal images showing Iba1 microglia (upper panels) and GFAP astrocytes (lower panels) in the ventral horn of lumbar spinal cord of non-transgenic, vehicle- and BANA-treated SOD1^{G93A} rats. The graphs to the right show quantitative analysis of microgliosis and astrogliosis in the ventral spinal cord. Data are expressed as mean±SEM; data were analyzed by the Kruskal-Wallis test followed by Dunn's multiple comparisons test, ****p<0.0001, *p=0.0104. n=4 animals per condition. Scale bars=50 μm.

FIG. 15C: Representative confocal images of ChAT-positive motor neurons in the ventral horn of lumbar spinal cord of non-transgenic, vehicle- and BANA-treated SOD1^{G93A} rats. The graph to the right of the images represents the quantitative analysis of the number of motor neurons in the ventral horn among conditions. All data are expressed as mean±SEM; data were analyzed by the Kruskal-Wallis test followed by Dunn's multiple comparisons test, ****p<0.0001. n=4 animals per condition. Scale bar=50 μm.

FIG. 16A: Representative confocal images from immunohistochemistry analysis of NF-κB-p65 nuclear expression (green) in Isolectin-positive microglia in the degenerating spinal cord of SOD1^{G93A} symptomatic rats. Upper panels show microglia displaying nuclear NF-κB-p65 (magenta arrow). Lower panels show higher magnification confocal images showing nuclear NF-κB-p65 (white arrowhead) expression in Isolectin-positive microglia (magenta) surrounding Nissl-positive motor neurons (white). The two panels to the right show the orthogonal visualization of nuclear (DAPI, red) localization of NF-κB-p65 (green). Scale bars=20 μm (upper panels) and 5 μm (lower panels).

FIG. 16B: Representative confocal images from immunohistochemistry analysis of NF-κB-p65 nuclear expression (green) in Iba1-positive microglia in the lumbar spinal cord of ALS subjects. Upper panels show NF-κB-p65 nuclear expression (green) in microglia (white) in the lumbar spinal cord of an ALS subject (magenta arrows). Lower panels show higher magnification images showing nuclear and cytoplasmic expression of NF-κB-p65 (green) in ALS-associated microglia clusters (magenta arrows) in the lumbar spinal cord of an ALS subject. Scale bars=20 μm.

FIG. 17A: Scheme of the experimental design in which NF-kB-RE-Luc tg mice were treated with 100 mg/kg/day of BANA or vehicle starting immediately before sciatic nerve section (day 0) until day 4.

FIG. 17B: Representative images obtained from in vivo luminescence imaging of NF-kB activation in the right hindlimb after sciatic nerve section in mice treated with vehicle or BANA. White dotted line in the upper left-hand image (day 1) represents the area of the hindlimb where luminescence was quantified. The graph to the right of FIG. 17B shows the quantitative analysis of NF-kB luminescence for each condition over time, and shows that BANA significantly prevented NFkB activation over time when compared with vehicle. All quantitative data are expressed as mean±SEM; data were analyzed by Two-way ANOVA followed by Sidak's multiple comparison test, ***p=0.0003.

### BRIEF SUMMARY OF THE INVENTION

An embodiment of the present invention relates to electrophilic nitroalkene derivatives, including nitroalkene aromatic acid derivatives, for the treatment of amyotrophic lateral sclerosis and related neurodegenerative conditions.

One embodiment includes a method of treating a neurodegenerative condition in a mammal comprising administering an effective amount of a nitroalkene derivative, such as a nitroalkene aromatic acid derivative, to the mammal. In one embodiment the neurodegenerative condition includes Alzheimer's Disease, Parkinson's Disease, multiple sclerosis, Huntington's Disease, amyotrophic lateral sclerosis (ALS), spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sach's, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy). In another embodiment the neurodegenerative disorder treated is preferably Alzheimer's Disease, Parkinson's Disease, multiple sclerosis, Huntington's Disease, and amyotrophic lateral sclerosis (ALS). In another embodiment the neurodegenerative condition treated is amyotrophic lateral sclerosis (ALS).

One embodiment includes a method of treating a neurodegenerative condition, wherein the nitroalkene derivative is a nitroalkene aromatic acid derivative. In another embodiment the nitroalkene derivative is preferably (E)-4-(2-nitrovinyl) benzoic acid.

Another embodiment includes a method of treating a neurodegenerative condition in a mammal comprising administering a pharmaceutical composition comprising an effective amount of a nitroalkene derivative and at least one pharmaceutically acceptable excipient to the mammal. In another embodiment, the neurodegenerative condition includes Alzheimer's Disease, Parkinson's Disease, multiple sclerosis, Huntington's Disease, amyotrophic lateral sclerosis (ALS), spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sach's, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy). In another embodiment the neurodegenerative condition treated is preferably Alzheimer's Disease, Parkinson's Disease, multiple sclerosis, Huntington's Disease, and amyotrophic lateral sclerosis (ALS). In yet another embodiment, the neurodegenerative condition treated is preferably is amyotrophic lateral sclerosis (ALS).

In one embodiment the pharmaceutical composition includes a nitroalkene derivative that is a nitroalkene aromatic acid derivative. In another embodiment, the pharmaceutical composition includes a nitroalkene derivative that is (E)-4-(2-nitrovinyl) benzoic acid. In one embodiment the present invention includes a method of treating a neurodegenerative condition using a nitroalkene aromatic acid derivative. In some embodiments, the present invention is directed to the use of a nitroalkene derivative to treat a neurodegenerative condition. In some embodiments, the nitroalkene derivative is preferably (E)-4-(2-nitrovinyl) benzoic acid.

One embodiment includes the use of a nitroalkene derivative for the preparation of a medicament for treating a mammal having a neurodegenerative condition. In another embodiment, the neurodegenerative condition includes Alzheimer's Disease, Parkinson's Disease, multiple sclerosis, Huntington's Disease, amyotrophic lateral sclerosis (ALS), spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sach's, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy). In another embodiment the neurodegenerative condition treated is preferably Alzheimer's Disease, Parkinson's Disease, multiple sclerosis, Huntington's Disease, and amyotrophic lateral sclerosis (ALS). In yet another embodiment, the neurodegenerative condition treated is preferably is amyotrophic lateral sclerosis (ALS).

One embodiment includes the use of a nitroalkene derivative for the preparation of a medicament for treating a mammal having a neurodegenerative condition, wherein the nitroalkene derivative is a nitroalkene aromatic acid derivative. In another embodiment, the nitroalkene aromatic acid derivative is (E)-4-(2-nitrovinyl) benzoic acid.

Use of a nitroalkene derivative for improving motor deficits in a mammal having a neurodegenerative condition. In another embodiment, the neurodegenerative condition includes Alzheimer's Disease, Parkinson's Disease, multiple sclerosis, Huntington's Disease, amyotrophic lateral sclerosis (ALS), spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sach's, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy). In another embodiment the neurodegenerative condition treated is preferably Alzheimer's Disease, Parkinson's Disease, multiple sclerosis, Huntington's Disease, and amyotrophic lateral sclerosis (ALS). In yet another embodiment, the neurodegenerative condition treated is preferably is amyotrophic lateral sclerosis (ALS).

Another embodiment includes the use of a nitroalkene derivative for improving motor deficits in a mammal having a neurodegenerative condition, wherein the nitroalkene derivative is a nitroalkene aromatic acid derivative. In another embodiment, the nitroalkene aromatic acid derivative is (E)-4-(2-nitrovinyl) benzoic acid.

One embodiment includes the use of a nitroalkene derivative for reducing neuroinflammation in a mammal having a neurodegenerative condition. In another embodiment, the neurodegenerative condition includes Alzheimer's Disease, Parkinson's Disease, multiple sclerosis, Huntington's Disease, amyotrophic lateral sclerosis (ALS), spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sach's, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy). In another embodiment the neurodegenerative condition treated is preferably Alzheimer's Disease, Parkinson's Disease, multiple sclerosis, Huntington's Disease, and amyotrophic lateral sclerosis (ALS). In yet another embodiment, the neurodegenerative condition treated is preferably is amyotrophic lateral sclerosis (ALS).

Another embodiment includes the use of a nitroalkene derivative for reducing neuroinflammation in a mammal having a neurodegenerative condition, wherein the nitroalkene derivative is a nitroalkene aromatic acid derivative. In another embodiment, the nitroalkene aromatic acid derivative is (E)-4-(2-nitrovinyl) benzoic acid.

Another embodiment includes the use of a nitroalkene derivative for reducing the release of IL-1β in a mammal having a neurodegenerative condition. In another embodiment, the neurodegenerative condition includes Alzheimer's Disease, Parkinson's Disease, multiple sclerosis, Huntington's Disease, amyotrophic lateral sclerosis (ALS), spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sach's, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy). In another embodiment the neurodegenerative condition treated is preferably Alzheimer's Disease, Parkinson's Disease, multiple sclerosis, Huntington's Disease, and amyotrophic lateral sclerosis (ALS). In yet another embodiment, the neurodegenerative condition treated is preferably is amyotrophic lateral sclerosis (ALS).

One embodiment includes the use of a nitroalkene derivative for reducing the release of IL-1β in a mammal having a neurodegenerative condition, wherein the nitroalkene derivative is a nitroalkene aromatic acid derivative. In another embodiment, the nitroalkene aromatic acid derivative is (E)-4-(2-nitrovinyl) benzoic acid.

Another embodiment includes the use of a nitroalkene derivative to downregulate NF-κB in a mammal having a neurodegenerative condition. In another embodiment, the neurodegenerative condition includes Alzheimer's Disease, Parkinson's Disease, multiple sclerosis, Huntington's Disease, amyotrophic lateral sclerosis (ALS), spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sach's, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy). In another embodiment the neurodegenerative condition treated is preferably Alzheimer's Disease, Parkinson's Disease, multiple sclerosis, Huntington's Disease, and amyotrophic lateral sclerosis (ALS). In yet another embodiment, the neurodegenerative condition treated is preferably is amyotrophic lateral sclerosis (ALS).

One embodiment includes the use of a nitroalkene to downregulate NF-κB in a mammal having a neurodegenerative condition, wherein the nitroalkene derivative a nitroalkene aromatic acid derivative. In some embodiments, the nitroalkene aromatic acid derivative is (E)-4-(2-nitrovinyl) benzoic acid.

### DETAILED DESCRIPTION OF THE INVENTION

The terminology used in the description is for the purpose of describing the particular versions or embodiments only, and is not intended to limit the scope of the present invention which will be limited only by the appended claims.

Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described. Nothing herein is to be construed as an admission that the invention is not entitled to antedate such disclosure by virtue of prior invention.

As used herein and in the appended claims, the singular forms “a,” “an,” and “the” include plural reference unless the context clearly dictates otherwise. Thus, for example, reference to a “cell” is a reference to one or more cells and equivalents thereof known to those skilled in the art, and so forth.

“Administering” when used in conjunction with a therapeutic means to deliver a therapeutic agent, such as in the case of the present invention, a nitroalkene derivative to a subject to provide a physiochemical effect. In some embodiments, administering the therapeutic agent to a subject provides a benefit, such as a clinically meaningful benefit to a subject in need thereof. “Administering” a composition may be accomplished by, for example, injection, oral administration, topical administration, or by these methods in combination with other known techniques. Such combination techniques include heating, radiation, ultrasound and the use of delivery agents. When a compound is provided in combination with one or more other active agents (e.g. other anti-atherosclerotic agents such as the class of statins), “administration” and its variants are each understood to include concurrent and sequential provision of the compound or salt and other agents.

By “pharmaceutically acceptable” it is meant the carrier, diluent, adjuvant, or excipient must be compatible with the other ingredients of the formulation and not deleterious to the recipient thereof.

“Composition” as used herein is intended to encompass a product comprising the specified ingredients in the specified amounts, as well as any product which results, directly or indirectly, from combination of the specified ingredients in the specified amounts. Such term in relation to “pharmaceutical composition” is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound o the present invention and a pharmaceutically acceptable carrier.

As used herein, the term “agent,” “active agent,” “therapeutic agent,” or “therapeutic” means a compound or composition utilized to treat, combat, ameliorate, prevent or improve an unwanted condition or disease of a patient. Furthermore, the term “agent,” “active agent,” “therapeutic agent,” or “therapeutic” encompasses a combination of one or more of the compounds of the present invention.

A “therapeutically effective amount” or “effective amount” of a composition is a predetermined amount calculated to achieve a desired effect in a subject. For example, a desired effect can be to inhibit, block, or reverse the activation, migration, proliferation, alteration of cellular function, and to preserve the normal function of cells. The activity contemplated by the methods described herein includes both medical therapeutic and/or prophylactic treatment, as appropriate, and the compositions of the invention may be used to provide improvement in any of the conditions described. It is also contemplated that the compositions described herein may be administered to healthy subjects or individuals not exhibiting symptoms but who may be at risk of developing a particular disorder. The specific dose of a compound administered according to this invention to obtain therapeutic and/or prophylactic effects will, of course, be determined by the particular circumstances surrounding the case, including, for example, the compound administered, the route of administration, and the condition being treated. However, it will be understood that the chosen dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The terms “treat,” “treated,” or “treating” as used herein refer to both therapeutic treatment and prophylactic or preventative measures, wherein the object is to prevent or slow down (lessen) an undesired physiological condition, disorder, or disease, or to obtain beneficial or desired clinical results. For the purposes of this invention, beneficial or desired results include, but are not limited to, alleviation of symptoms; diminishment of the extent of the condition, disorder, or disease; stabilization (i.e., not worsening) of the state of the condition, disorder, or disease; delay in onset or slowing of the progression of the condition, disorder, or disease; amelioration of the condition, disorder, or disease state; and remission (whether partial or total), whether detectable or undetectable, or enhancement or improvement of the condition, disorder, or disease. Treatment includes prolonging survival as compared to expected survival if not receiving treatment.

Motor neuron degeneration and neuroinflammation are the most striking pathological features of ALS. Within the scope of the described inventions are synthesized nitroalkene derivatives that are bioavailable when administered orally to rats. In cell cultures, the nitroalkene derivatives were devoid of toxicity at low micromolar concentrations and exerted a potent anti-inflammatory effect in myeloid and microglia cell lines, inhibiting LPS-induced NLRP3 inflammasome activation and NF-κB signaling. In microglia cells isolated from symptomatic SOD1^{G93A} rats, the nitroalkene derivatives potently inhibited cell proliferation and phenotypic transformation into neurotoxic aberrant cells, as well as LPS-induced NF-κB p65 nuclear translocation. For example, E)-4-(2-nitrovinyl) benzoic acid (BANA) exerted a potent anti-inflammatory effect in myeloid and microglia cell lines, and prolonged post-paralysis survival by 32% respect to vehicle when orally administered to SOD1^{G93A} rats starting after disease onset. Compared to the control vehicle, BANA-treated rats displayed preserved number and size of spinal motor neurons, and decreased microgliosis and astrocytosis in the lumbar spinal cord. These data provide a rationale to therapeutically delay paralysis progression in ALS using small electrophilic compounds such as BANA, through simultaneous modulation of inflammatory pathways. Remarkably and unlike many other drugs that have been tested in mutant SOD1 animal models, the BANA's protective effects were observed when the treatment was initiated after paralysis onset, making it well adapted to the clinical setup of ALS patients.

Accordingly, within the scope of the described inventions is a new drug candidate for the treatment of ALS and related neurodegenerative diseases where neuroinflammation contributes to neuronal degeneration and to the ineluctable progression of neurological deficits that characterize neurodegenerative diseases. Without wishing to be bound by theory, the compounds described within the scope of the inventions allow the control of systemic inflammation mediated by immune cells, neuroinflammation mediated by glial cells in the CNS and cytoprotection of many different cell types supporting the neuromuscular function, including motor neurons, myocytes, Schwann cells, etc.

### Administration and Compositions

The pharmaceutical compositions included within the scope of the present invention comprise a therapeutically effective amount Compound 1 and at least one pharmaceutically acceptable excipient. The term “excipient” refers to a pharmaceutically acceptable, inactive substance used as a carrier for the pharmaceutically active ingredient (Compound 1), and includes anti adherents, binders, coatings, disintegrants, fillers, diluents, solvents, flavors, bulkants, colours, glidants, dispersing agents, wetting agents, lubricants, preservatives, sorbents and sweeteners. The choice of excipient(s) will depend on factors such as the particular mode of administration and the nature of the dosage form. Solutions or suspensions used for injection or infusion can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes, including autoinjectors, or multiple dose vials made of glass or plastic.

In the methods of various embodiments, pharmaceutical compositions including the active agent can be administered to a subject in an “effective amount” or “therapeutically effective amount,” which may be any amount that provides a beneficial effect to the subject.

A pharmaceutical formulation of the present invention may be in any pharmaceutical dosage form. The pharmaceutical formulation may be, for example, a tablet, capsule, nanoparticulate material, e.g., granulated particulate material or a powder, a lyophilized material for reconstitution, liquid solution, suspension, emulsion or other liquid form, injectable suspension, solution, emulsion, etc., suppository, or topical or transdermal preparation or patch. The pharmaceutical formulations generally contain about 1% to about 99% by weight of Compound 1 and 99% to 1% by weight of a suitable pharmaceutical excipient. In one embodiment, the dosage form is an oral dosage form. In another embodiment, the dosage form is a parenteral dosage form. In another embodiment, the dosage form is an enteral dosage form. In another embodiment, the dosage form is a topical dosage form. In one embodiment, the pharmaceutical dosage form is a unit dose. The term ‘unit dose’ refers to the amount of Compound 1 administered to a patient in a single dose.

A pharmaceutical formulation may be, for example, an oral dosage form for controlled release. By way of example only, controlled or modified release oral dosage forms can be prepared by using methods known in the art. For example, a suitable controlled release form of Compound I may be a matrix tablet or a capsule dosage composition. Suitable materials for matrix dosage forms include, for example, waxes (e.g. carnauba, bees wax, paraffin wax, ceresine, shellac wax, fatty acids, and fatty alcohols), oils, hardened oils or fats (e.g., hardened rapeseed oil, castor oil, beef tallow palm oil, and soya bean oil), and polymers (e.g., hydroxypropyl cellulose, polyvinylpyrrolidone, hydroxypropyl methyl cellulose, and polyethylene glycol). Other suitable matrix tableting materials include microcrystalline cellulose, powdered cellulose, hydroxypropyl cellulose, ethyl cellulose, with other carriers, and fillers. Tablets may also contain granulates, coated powders, or pellets. Tablets may also be multi-layered. Multi-layered tablets are especially preferred when the active ingredients have markedly different pharmacokinetic profiles. The finished tablet may also be coated or uncoated.

The coating composition typically contains an insoluble matrix polymer (approximately 15-85% by weight of the coating composition) and a water soluble material (e.g., approximately 15-85% by weight of the coating composition). Optionally an enteric polymer (approximately 1 to 99% by weight of the coating composition) may be used or included. Suitable water soluble materials include polymers such as polyethylene glycol, hydroxypropyl cellulose, hydroxypropyl methyl cellulose, polyvinylpyrrolidone, polyvinyl alcohol, and monomeric materials such as sugars (e.g., lactose, sucrose, fructose, mannitol and the like), salts (e.g., sodium chloride, potassium chloride and the like), organic acids (e.g., fumaric acid, succinic acid, lactic acid, and tartaric acid), and mixtures thereof. Suitable enteric polymers include hydroxypropyl methyl cellulose, acetate succinate, hydroxypropyl methyl cellulose, phthalate, polyvinyl acetate phthalate, cellulose acetate phthalate, cellulose acetate trimellitate, shellac, zein, and polymethacrylates containing carboxyl groups. The coating composition may be plasticised according to the properties of the coating blend such as the glass transition temperature of the main component or mixture of components or the solvent used for applying the coating compositions. Suitable plasticisers may be added from 0 to 50% by weight of the coating composition and include, for example, diethyl phthalate, citrate esters, polyethylene glycol, glycerol, acetylated glycerides, acetylated citrate esters, dibutylsebacate, and castor oil. If desired, the coating composition may include a filler. The amount of the filler may be 1% to approximately 99% by weight based on the total weight of the coating composition and may be an insoluble material such as silicon dioxide, titanium dioxide, talc, kaolin, alumina, starch, powdered cellulose, MCC, or polacrilin potassium. The coating composition may be applied as a solution or latex in organic solvents or aqueous solvents or mixtures thereof. If solutions are applied, the solvent may be present in amounts from approximate by 25-99% by weight based on the total weight of dissolved solids. Suitable solvents are water, lower alcohol, lower chlorinated hydrocarbons, ketones, or mixtures thereof. If latexes are applied, the solvent is present in amounts from approximately 25-97% by weight based on the quantity of polymeric material in the latex. The solvent may be predominantly water.

In some embodiments, a pharmaceutical composition of the present invention is delivered to a subject via a parenteral route, an enteral route, or a topical route. Examples of parental routes the present invention include, without limitation, any one or more of the following: intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumoral, intratympanic, intrauterine, intravascular, intravenous (bolus or drip), intraventricular, intravesical, and/or subcutaneous.

Enteral routes of administration of the present invention include administration to the gastrointestinal tract via the mouth (oral), stomach (gastric), and rectum (rectal). Gastric administration typically involves the use of a tube through the nasal passage (NG tube) or a tube in the esophagus leading directly to the stomach (PEG tube). Rectal administration typically involves rectal suppositories. Oral administration includes sublingual and buccal administration.

Topical administration includes administration to a body surface, such as skin or mucous membranes, including intranasal and pulmonary administration. Transdermal forms include cream, foam, gel, lotion or ointment. Intranasal and pulmonary forms include liquids and powders, e.g., liquid spray.

Further guidance for methods suitable for use in preparing pharmaceutical compositions is provided in Remington: The Science and Practice of Pharmacy, 21^{st} edition (Lippincott Williams & Wilkins, 2006).

The dose may vary depending upon the dosage form employed, sensitivity of the patient, and the route of administration. Dosage and administration are adjusted to provide sufficient levels of the active agent(s) or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy.

In one embodiment, the daily dose of a nitroalkene derivative, for example BANA, administered to a patient is selected from: up to 200 mg, 175 mg, 150 mg, 125 mg, 100 mg, 90 mg, 80 mg, 70 mg, 60 mg, 50 mg, 30 mg, 25 mg, 20 mg, 15 mg, 14 mg, 13 mg, 12 mg, 11 mg, 10 mg, 9 mg, 8 mg, 7 mg, 6 mg, 5 mg, 4 mg, 3 mg, 2 mg, 1 mg, 0.5 mg, or up to 0.1 mg.

In another embodiment, the daily dose is at least 0.05 mg, 0.1 mg, 0.5 mg, 1 mg, 2 mg, 3 mg, 4 mg, 5 mg, 6 mg, 7 mg, 8 mg, 9 mg, 10 mg, 12 mg, 13 mg, 14 mg, 15 mg, 20 mg, 25 mg, 30 mg, 40 mg, 50 mg, 60 mg, 70 mg, 80 mg, 90 mg, 100 mg, 125 mg, 150 mg, 175 mg, or at least 200 mg. In another embodiment, the daily dose is 0.05-1 mg, 1-2 mg, 2-4 mg, 1-5 mg, 5-7.5 mg, 7.5-10 mg, 10-15 mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, or 45-50 mg, 50-75 mg, 75-100 mg, 100-125 mg, 125-150 mg, 150-175 mg, 175-200 mg, or more than 200 mg.

In another embodiment, a single dose of a nitroalkene derivative, for example BANA, administered to a patient is selected from about: 0.05 mg, 0.1 mg, 0.5, mg, 1 mg, 2 mg, 3 mg, 4 mg, 5 mg, 6 mg, 7 mg, 8 mg, 9 mg, 10 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, or 50 mg.

In another embodiment, a single dose of a nitroalkene derivative, for example BANA, administered to a patient is selected from about: 0.05-1 mg, 1-2 mg, 2-4 mg, 1-5 mg, 5-7.5 mg, 7.5-10 mg, 10-15 mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, 45-50 mg, 50-75 mg, 75-100 mg, 100-125 mg, 125-150 mg, 150-175 mg, 175-200 mg, or more than 200 mg.

In one embodiment, the single dose is administered by a route selected from any one of: oral, buccal, or sublingual administration. In another embodiment, said single dose is administered by injection, e.g., subcutaneous, intramuscular, or intravenous. In another embodiment, said single dose is administered by inhalation or intranasal administration.

As a non-limited example, the dose of the nitroalkene derivative may be administered by injection may be about 0.05 to 50 mg per day to be administered in divided doses. A single dose of Compound 1 administered by subcutaneous injection may be about 0.05-6 mg, preferably about 1-4 mg, 1-3 mg, or 2 mg. Infusion may be preferable in those patients requiring division of injections into more than 10 doses daily. The continuous subcutaneous infusion dose may be 1 mg/hour daily and is generally increased according to response up to 4 mg/hour.

Long-acting pharmaceutical compositions may be administered, 1, 2, 3, 4, 5, 6, 7, 8, 9, 10 or more than 10 times daily (preferably: 1 times per day), every other day, every 3 to 4 days, every week, or once every two weeks depending on half-life and clearance rate of the particular formulation.

Pharmaceutical compositions comprising a nitroalkene derivative, for example BANA, and pharmaceutically-acceptable salts thereof can be administered by means that produces contact of the active agent with the agent's site of action. They can be administered by conventional means available for use in conjunction with pharmaceuticals in a dosage range of 0.001 to 1000 mg/kg of mammal body weight per day in a single dose or in divided doses. One dosage range is 0.01 to 500 mg/kg body weight per day in a single dose or in divided doses.

Administration can be delivered as individual therapeutic agents or in a combination of therapeutic agents. They can be administered alone, but typically are administered with a pharmaceutically acceptable excipient selected on the basis of the chosen route of administration and standard pharmaceutical practice.

### Indications Suitable for Treatment

The pharmaceutical compositions of the present invention may be employed to treat or reduce the symptoms associated with systemic inflammation mediated by immune cells, neuroinflammation mediated by glial cells in the CNS, and cytoprotection of many different cell types supporting the neuromuscular function, including motor neurons, myocytes, Schwann cells, etc.

Because nitroalkene derivatives, for example BANA, exhibit a potent anti-inflammatory activity as assessed in macrophages and microglia cell cultures through inhibition of the inflammatory pathway NF-κB and activation of the cytoprotective pathway Nrf2/keap1 via electrophilic properties that have the ability to modulate Nrf2/keap1, NF-κB-pathways in a variety of target cells including astrocytes, nitroalkene derivatives described within the scope of the inventions here in are suitable for improving motor deficits and reducing neuroinflammation in various neurodegenerative conditions, for example ALS. The nitroalkene derivatives further exhibit anti-inflammatory effects associated with activation of PPAR-γ and the Heat Shock Response.

Conditions suitable for treatment according to this invention include neurodegenerative diseases include Alzheimer's disease, Parkinson's Disease, multiple sclerosis, Huntington's Disease, amyotrophic lateral sclerosis (ALS), spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sach's, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy).

### EXAMPLES

### Example 1: Synthesis of (E)-4-(2-nitrovinyl) benzoic acid (BANA)

The following examples contain detailed methods of preparing BANA. Synthesis is presented for illustrative purposes only and is not intended as a restriction on the scope of the invention. All parts are by weight and temperatures are in Degrees Celsius unless otherwise indicated.

4-Formylbenzoic acid (26.6 mmol), nitromethane (369.2 mmol), ammonium acetate (33.1 mmol) and acetic acid (30 mL) were sequentially introduced into a round bottom flask; then it was placed in a stirring oil bath preheated at 90° C. for 4 hours. The reaction mixture was then allowed to cool, the precipitate was isolated via filtration and wash with cold water. After drying the desired product was obtained as a yellow solid (82%). m.p. 270-272° C. (decomposition was observed). ¹H-NMR (acetone-d₆): δ=8.30 (d, J=13.6 Hz, 1H); 8.18 (d, J=13.6 Hz, 1H); 8.00 (d, J=8.4 Hz, 2H); 7.97 (d, J=8.4 Hz, 2H). ¹³C-NMR: δ=207.3; 167.1; 140.1; 138.3; 134.9; 133.8; 130.3. MS (IE, 70 eV): m/z (%)=193 (M+, 86), 148 (51), 121 (10), 102 (43), 91 (85), 77 (100).

Due to the coupling constant of the doublets of the alkene observed in the ¹H NMR spectra, the configuration of the nitroalkene was consistent with the E-isomer.

### Example 2: BANA Electrophilic Reactivity

Most electrophilic nitroalkene compounds exert their pharmacological activity via a Michael addition reaction with nucleophiles. To study the electrophilic properties of BANA, the reaction between -mercaptoethanol (BME) and BANA was studied spectrophotometrically.

The electrophilic reactivity of BANA compound was determined by analyzing the UV-Visible spectra of the reaction between BANA and β-Mercaptoethanol (β-ME). BANA (30 μM) was incubated with β-ME (300 μM) in Phosphate buffer (20 mM) pH=7.4, and scans were taken each min up to 15 min.

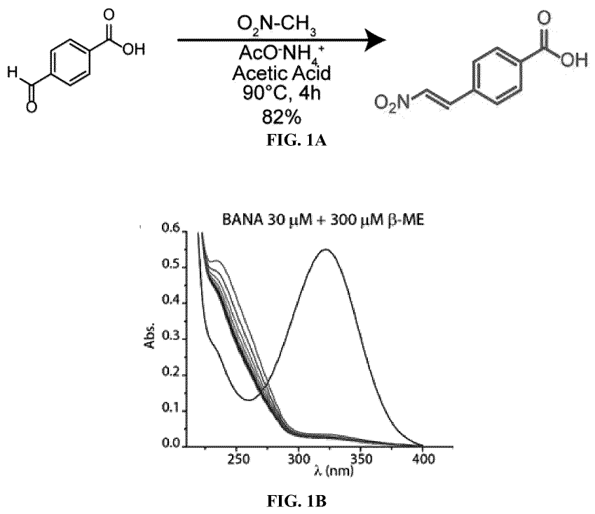

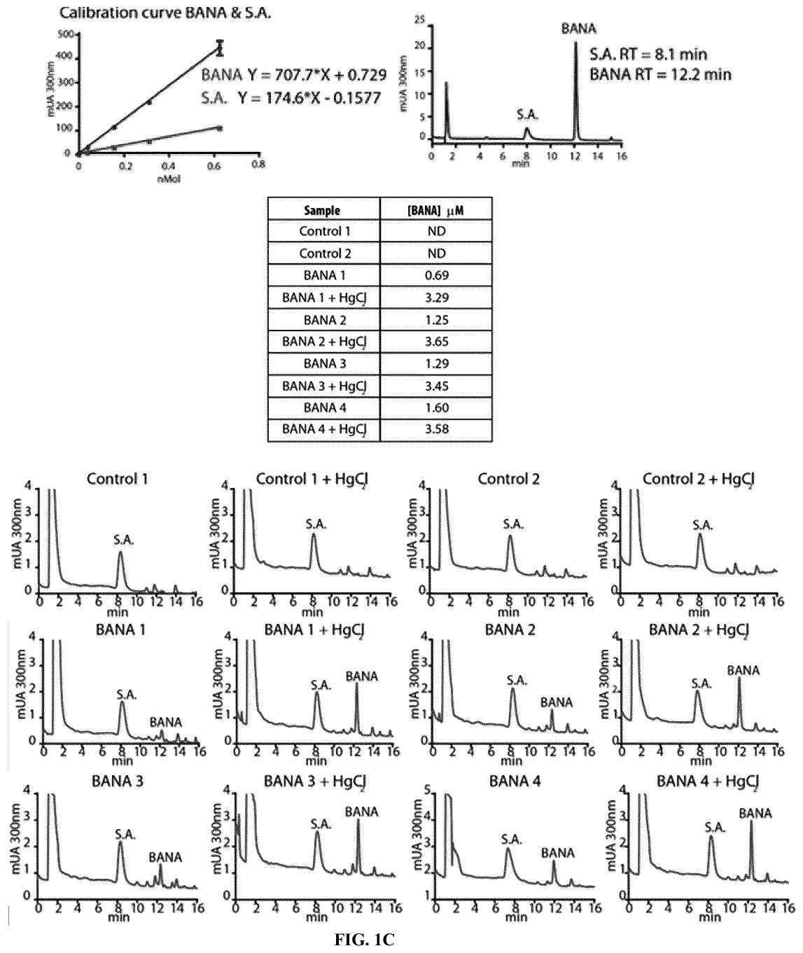

FIG. 1B shows the ability of BANA to react with 3-ME, resulting in a decreased in absorbance.

FIG. 1C shows plasma levels of BANA as measured by HPLC after oral administration of BANA to rats (100 mg/kg/day for 5 days). BANA was found in low micromolar concentrations, more than 90% was covalently linked to proteins.

### Example 3: Effect of BANA on Nuclear Translocation of NF-κB in Microglia

The effect of BANA on nuclear translocation of NF-κB was studied by immunocytochemistry using murine microglia cell line BV2.

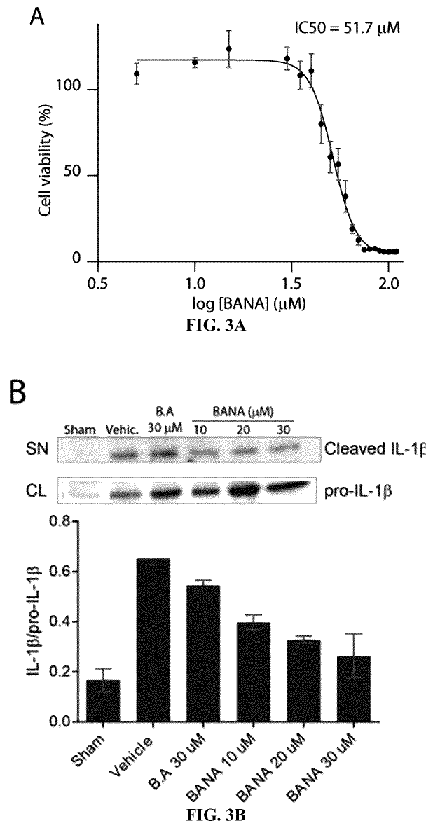

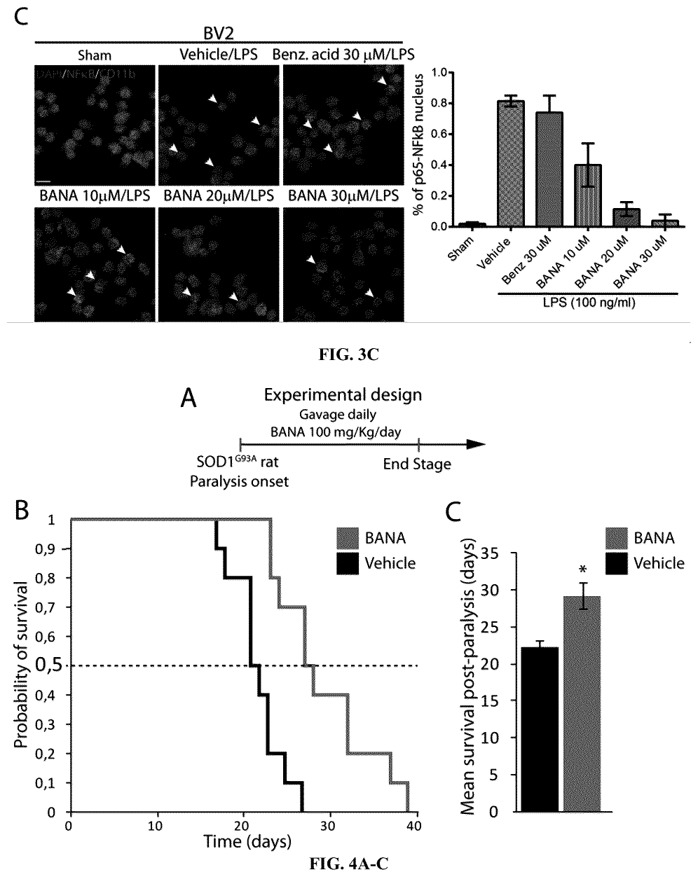

The murine BV2 microglial cell line was used to analyze the toxicity and anti-inflammatory effects of BANA. Baseline cell viability was established in murine microglia cell line BV2 by sulforhodamine B assay. Briefly, cells were plated in a 96-multiwell plate and incubated with different concentrations of BANA (5-110 μM) for 24 h. After media removal, cells were washed twice with PBS pH=7.4. Cells were fixed with TCA for 1 hour at 4° C. and then washed five times with distilled water. 50 μL of sulforhodamine B 0.4% m/v in 1% acetic acid was added to each well and incubated 30 minutes at room temperature. After staining, the plate was washed at least five times with 1% acetic acid. Once the plate was dry the protein-bound dye was dissolved in 10 mM Tris and the absorbance at 570 nm was read using a microplate spectrophotometer (FIG. 3A).

Cells were then treated with BANA (10, 20 and 30 μM) or B.A (30 μM) for 3 hrs. After treatment, cells were stimulated with LPS (100 ng/ml) for 30 minutes. Then, cells were washed and fixed with PFA 4% for 20 minutes at 4° C. and washed with PBS. Immunocytochemistry was performed as follows: cells were permeabilized using 0.5% Triton in PBS for 15 minutes, and then blocked using 5% of BSA for 1 h at room temperature. Rabbit anti-NF-κB-p65 (ab16502, 1/200) and mouse anti-CD11b (BD550219, 1/100) were incubated overnight at 4° C. After that, primary antibodies were removed, washed with PBS 3 times for 10 minutes, and Alexa-fluor-conjugated goat anti-rabbit or goat anti-mouse antibodies (1:1000) were incubated for 2 h at room temperature. After secondary antibodies were removed, cells were covered in glycerol mounting medium with 1/2000 DAPI staining. Cells were analyzed by confocal microscopy using a confocal ZEISS LSM 800. NF-κB-p65+ nuclei were counted and ratio of DAPI to NF-κB-p65 labeling was compared among groups. Data were analyzed using analyzing tools of ImageJ and GraphPad Prism 7 software.

As shown in FIG. 3A, BANA was devoid of toxicity to BV2 microglia in low micromolar concentrations, the IC₅₀ being 51.7 μM. The results show that BANA downregulates microglia inflammatory response.

BANA also inhibited LPS/ATP-induced inflammasome activation in BV2 cells estimated by the release of IL1-β adjusted to pro-IL-1 β levels (FIG. 3B), as compared to vehicle or sodium benzoate. Also, BANA potently prevented the NF-κB p65 nuclear translocation induced 3 hours after exposure to LPS in BV2 cells (FIG. 3C), further suggesting a potent anti-inflammatory activity in microglia cells.

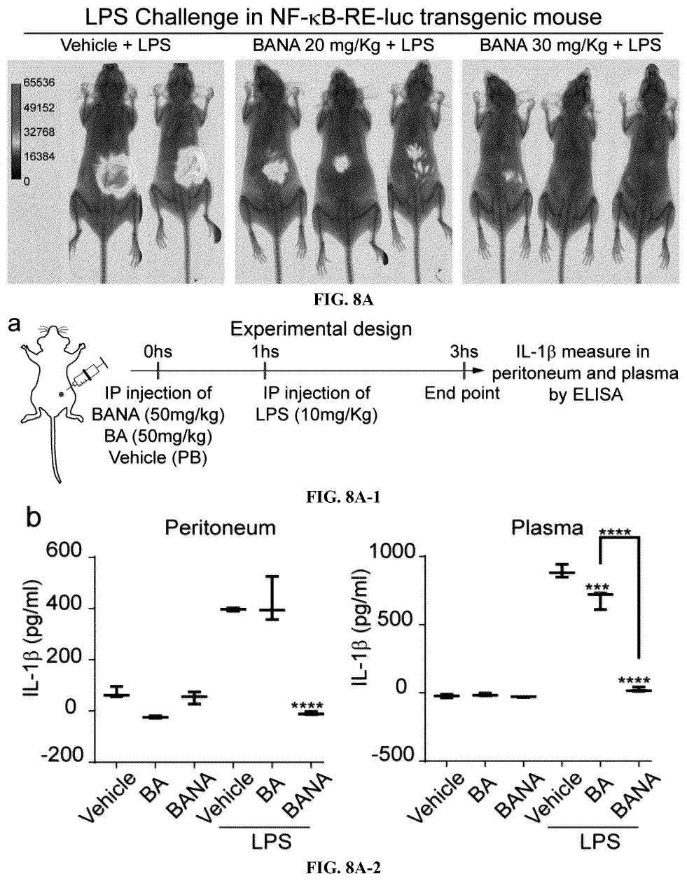

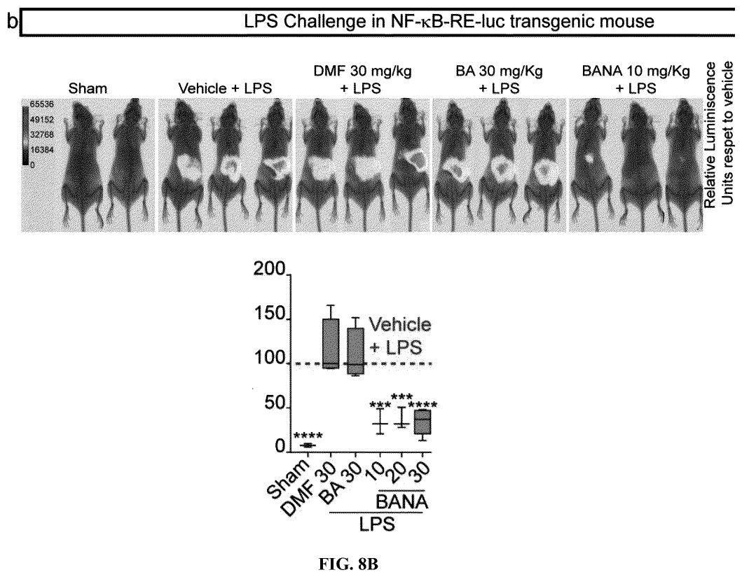

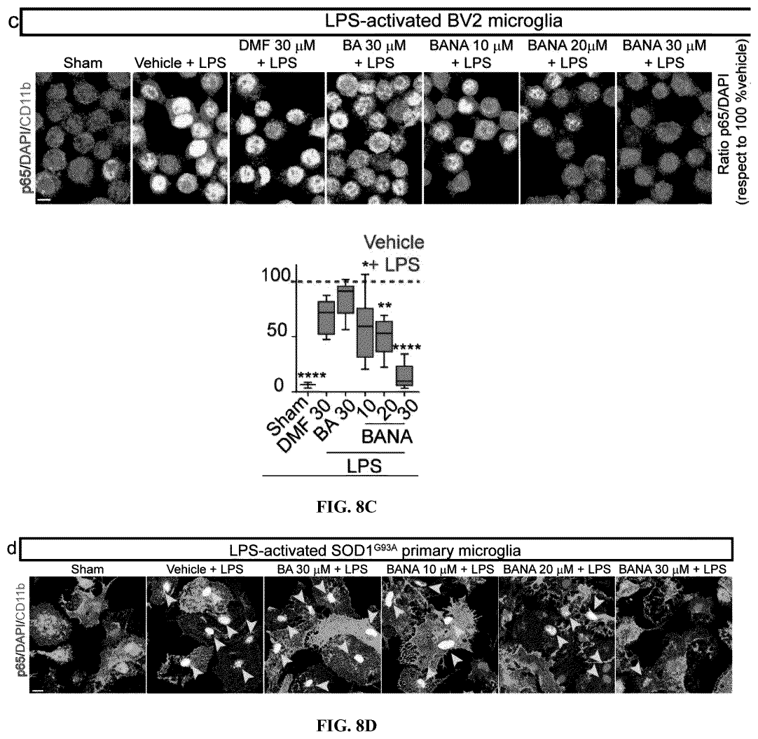

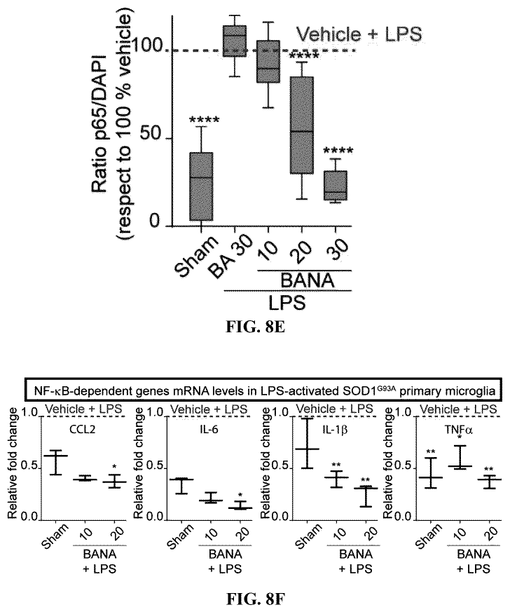

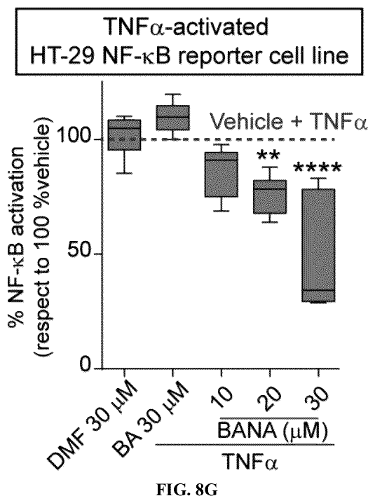

Confocal images of BV2 microglia treated with BANA, BA, or DMF before LPS stimulation (FIG. 8C) show that BANA inhibits LPS-induced NF-κB-p65 nuclear translocation.

Cells in the sham condition were not treated with LPS. Nuclear NF-κB-p65 colocalizing with DAPI nuclear staining is denoted in yellow. The graph in FIG. 8C shows the quantitative analysis of the ratio nuclear NF-κB-p65/DAPI among experimental conditions. All quantitative data are expressed as mean±SEM; data were analyzed by Ordinary one-way ANOVA followed by Tukey's multiple comparisons test, *p=0.0148 **p=0.0031 ****p<0,0001. 3 replicates of 3 independent experiments. Scale bar=10 μm.

### Example 4: Effect of BANA on NF-κB-Dependent Gene Expression

The effect of BANA or BA over NF-κB-dependent gene expression was studied by qPCR using THP-1 cells. THP-1 cells were differentiated into macrophages and treated with BANA (30 μM) or BA (30 μM) for 2 hs, followed by stimulation with LPS (100 ng/mL, for 3-4 h).

For inflammasome activation, THP-1 cells were treated with LPS (as above) followed by exposure to ATP (5 mM, 45 min). IL-1β in the supernatant was then measured by ELISA (B&D OptEIA TM, San Diego, CA, USA).

TNFα, CCL2, IL6 and IL-1β expression was analyzed by RT-PCR. Real-time PCR analysis was conducted as follows: Total mRNA was extracted from THP-1 cells to quantify TNFα, IL-6 and MCP-1-fold change gene expression over control (β-actin) by qPCR assay. Purified RNA was transcripted to cDNA using Superscript II Reverse Transcriptase with Oligo (dT). The cDNA for real-time PCR was obtained with a Piko 24 Thermal Cycler (Thermo Scientific). mRNA expression analysis was calculated using the ddCt method with β-Actin as the house keeping gene. SYBR Green was used as DNA binding dye and the qRT-PCR was done in an Eco Illumina thermocycler. Human primers sequences were used for THP-1 cells:

β-Actin forward:
5′-CATGTACGTTGCTATCCAGGC-3′;
β-Actin reverse:
5′-CTCCTTAATGTCACCCACGAT-3′;
TNFα forward:
5′-ATC CGA GAT GTG GAA CTG GC-3′
TNFα reverse:
5′-TGG GAA CTT CTC CTC CTT GTT G-3′
IL-6 forward:
5′-AGTGAGGAACAAGCCAGAGC-3′;
IL-6 reverse:
5′-ATTTGTGGTTGGGTCAGGGG-3′;
MCP-1 forward:
5′-CATAGCAGCCACCTTCATTCC-3′;
and
MCP-1 reverse:
5′-TCTCCTTGGCCACAATGGTC-3′.

### Effect of BANA on Inflammasome Activation

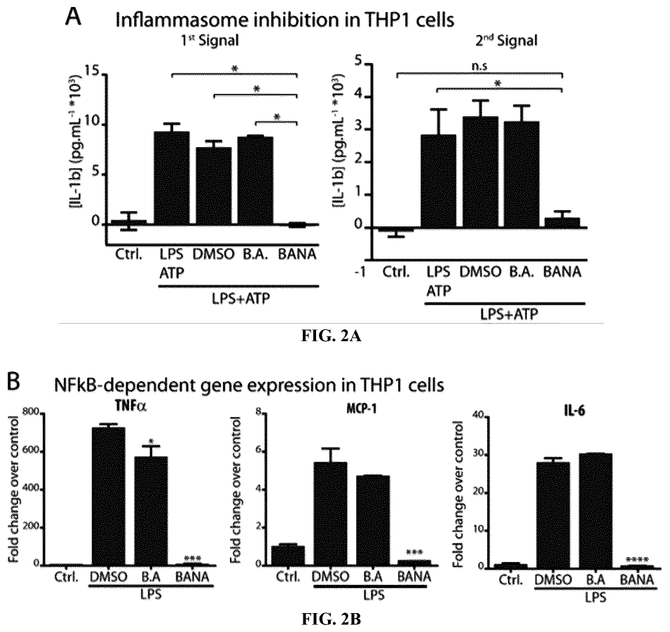

As shown in FIG. 2A, treatment of THP-1 cells with BANA (30 μM) in the first or second signal (LPS and ATP respectively), almost completely prevented the release of IL-1β as compared with vehicle. In contrast, sodium benzoate (30 μM) was devoid of anti-inflammatory effects. This result suggests BANA potently interferes with NLRP3 inflammasome activation.

The inventors also analyzed whether BANA could also downregulate NF-κB. As shown in FIG. 2B, BANA potently prevented the levels of mRNA coding for TNFα, MCP-1 and IL-6 respect to vehicle or sodium benzoate. Because these inflammatory genes are mainly regulated by NF-κB activation, this result suggests another mechanism by which BANA exerts anti-inflammation in macrophages. In sum, these results demonstrate that BANA exerts potent anti-inflammatory activity in THP-1 macrophage cell lines.

### Examples in Animal Models

### Experimental Background

Male SOD1^{G93A} progeny were used for further breeding to maintain the line (Taconic Biosciences, Inc, NTac:SD-Tg(SOD1^{G93A}) L26H). Rats were housed in a centralized animal facility with a 12-h light-dark cycle with ad libitum access to food and water. Perfusion with fixative was performed under 90% ketamine-10% xylazine anesthesia and all efforts were made to minimize animal suffering, discomfort or stress. All procedures using laboratory animals were performed in accordance with the national and international guidelines and were approved by the Institutional Animal Committee for animal experimentation. This study was carried out in strict accordance with the Institut Pasteur de Montevideo Committee's requirements and under the current ethical regulations of the Uruguayan Law No 18.611 for animal experimentation that follows the Guide for the Care and Use of Laboratory Animals of the National Institutes of Health (USA).

Only female transgenic rats showing weakness and gait alterations in the forelimbs as the first clinical sign were selected for BANA treatment studies. Rats were divided randomly into BANA or vehicle-treated groups. BANA was freshly prepared in buffer phosphate (PB), and administrated daily at a dose of 100 mg/kg using a curved stainless steel gavage needle with 3-mm ball tip. Rats were treated from day-1 post-paralysis for 15 days or until end-stage when they were euthanized.

### Determination of Disease Onset and End-Stage

All rats were weighed and evaluated for motor activity daily. Disease onset was determined for each animal when pronounced muscle atrophy accompanied by abnormal gait, typically expressed as subtle limping or dragging of one hind limb. End-stage was defined by a lack of righting reflexes or the inability to reach food and water.

### Statistical Analysis and Survival Curves

Quantitative data were expressed as mean±SEM. Ordinary one-way ANOVA followed by Tukey's multiple comparisons test, Kruskal-Wallis followed by Dunn's multiple comparison test, Mann-Whitney comparisons test or unpaired t-test were used for statistical analysis, with p<0.05 considered significant. GraphPad Prism 7.03 software was used for statistical analyses. All selected images represent the mean value for each condition.

Survival curves were compared by Kaplan-Meier analysis with the Log-rank test using GraphPad Prism 7 software.

### Example 5: Determination of BANA Plasma Levels

For the detection of BANA in plasma, experiments were performed using RP-HPLC analysis (Agilent 1200 HPLC system). Rats were sacrificed and plasma samples were obtained. 100 μL of plasma was incubated with HgCl₂ (20 mM) at 37° C. for 30 minutes and after that extracted with 900 μL of acetonitrile (ACN) and injected into HPLC in a 20-40% gradient of B in 10 minutes. BANA plasma concentration was calculated from a standard curve using Salicylate as an internal standard. (CPAK C-18, 4 μM, 150 mm×3.9 mm I.D. column; A phase: H2O 0.1% Formic acid; B phase: ACN 0.1% Formic acid; detection at 300 nm)

Rates were dosed with 100 mg/kg/day of BANA administered orally. BANA plasma levels reached 3.49 μM as assessed by HPLC following 100 mg/kg/day dosing, as shown in the following Table 1.

TABLE 1
Measured BANA concentrations in plasma of SOD^{1G93}A
symptomatic rats after 15 days of oral daily treatment
with BANA (100 mg/kg) or vehicle. n = 4 BANA-
administered rats and 2 vehicle-administered rats.

| Plasma Samples of SOD1^{G93A} | |
|---|---|
| Symptomatic Treated Rats | [BANA] μm |
| Vehicle 1 | N.D. |
| Vehicle 2 | N.D. |
| BANA 1 | 0.69 |
| BANA 1 + HgCl₂ | 3.29 |
| BANA 2 | 1.25 |
| BANA 2 + HgCl₂ | 3.69 |
| BANA 3 | 1.29 |
| BANA 3 + HgCl₂ | 3.45 |
| BANA 4 | 1.60 |
| BANA 4 + HgCl₂ | 3.58 |

### Example 6: BANA to Prolong Survival in SOD1^{G93A} Rats

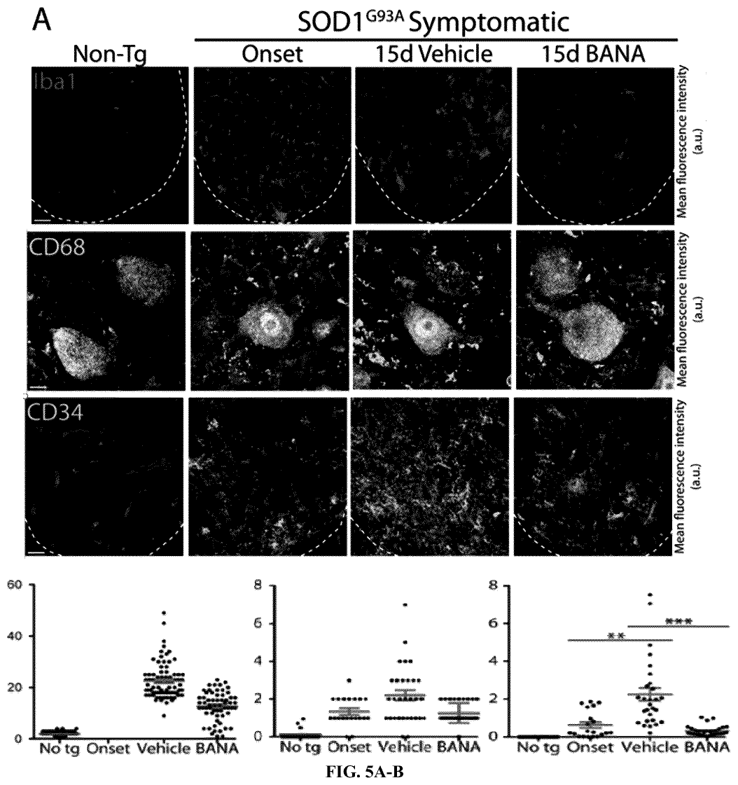

To test whether systemic treatment with BANA could prolong the survival of SOD1^{G93A} rats, a trial was conducted in which rats were orally treated with BANA (100 mg/kg/day) or vehicle, starting immediately after paralysis onset of one hind limb until the end-stage of the disease. Those rats that received vehicle died within three weeks after paralysis onset (FIG. 4A), while rats that received BANA had a 32% increase probability of survival compared with vehicle-treated rats (FIG. 4B). When the mean survival time of each condition was compared, there was evidence of a survival benefit for BANA (FIG. 4C).

### Example 7: BANA-Inhibition of LPS-Induced NF-κB Activation in Rats

To determine whether BANA was capable of preventing LPS-induced NF-κB activation, intraperitoneal administration of BANA (10, 20, and 30 mg/kg) were followed by LPS intraperitoneal administration to an NF-κB-reporter transgenic mouse. Its scaffold benzoic acid (30 mg/kg) and dimethyl fumarate (30 mg/kg), an electrophilic drug currently assayed in ALS clinical trials, were used as controls. BANA, Benzoic acid (BA), or dimethyl fumarate (DMF) were administered 2 h before intraperitoneal injection of LPS.

BANA treatment significantly decreased NF-κB activation pathway at all studied doses (FIGS. 8A and 8B). The graph in FIG. 8B shows the quantitative analysis in different experimental conditions. All quantitative data are expressed as mean±SEM; data were analyzed by Ordinary one-way ANOVA followed by Tukey's multiple comparisons test, ***p=0.0009 ****p<0,0001. n=4 animals per condition.

In comparison, the BA scaffold (30 mg/kg) and the electrophilic drug dimethyl fumarate (30 mg/kg) were devoid of inhibitory effect in NF-κB pathways in this model. In turn, BANA was able to reduce IL-1β concentration in plasma and peritoneum of mice intraperitoneally injected with LPS respect to controls (FIGS. 8A-1 and 8A-2). BANA (50 mg/kg), BA (50 mg/kg) or vehicle, were intraperitoneally (IP) injected 1 h before LPS intraperitoneal administration (10 mg/kg). 3 h later, IL-1β levels were measured in the peritoneum and plasma by ELISA. The data shows that BANA significantly reduces IL-1β concentration in the peritoneum (FIG. 8A-2, left graph) and plasma (FIG. 8A-2, right graph) when compared to vehicle and BA. All data are expressed as mean±SEM; data were analyzed by Ordinary one-way ANOVA followed by Tukey's multiple comparisons test, ***p=0.0002 ****p<0,0001. n=3 animals per condition.

### Example 8: Immunohistochemical Staining of Rat Spinal Cords

After 15 days of treatment using 100 mg/kg/day of BANA, starting after paralysis onset, animals were deeply anesthetized and transcardial perfusion was performed with 0.9% saline and 4% paraformaldehyde in 0.1 μM PBS (pH 7.2-7.4). Fixed spinal cords were removed, post-fixed by PFA 4% immersion overnight, and then transverse sectioned (30 m) in a Leica cryostat. Serial sections were collected in 100 mM PBS for immunohistochemistry.

At least 4 rats were analyzed for each immunohistochemistry experiment. Three different conditions were studied as follows: 1) non-transgenic (NonTg) rats of 160-180 days; 2) transgenic SOD1^{G93A} rats of 195-210 days treated with vehicle (paralysis, 15 d-vehicle) and 3) transgenic SOD1^{G93A} rats of 195-210 days treated with 50 or 100 mg/kg/day of BANA during 15 days. After treatment animals were deeply anesthetized and transcardial perfusion was performed with paraformaldehyde 4% (v/v) in PBS pH=7.4. Fixed spinal cords were removed, post-fixed by immersion overnight in paraformaldehyde 4% (v/v) in PBS pH=7.4, and then transverse sectioned (30 m) in a Leica cryostat. Serial sections were collected in PBS pH=7.4 for immunohistochemistry.

Free-floating sections were blocked and permeabilized for 1 hour at room temperature with 0.5% Triton X-100 and 5% BSA in PBS (pH=7.4), passed through washing buffered solutions, and incubated overnight at 4° C. in a solution of 0.5% Triton X-100 and BSA 1% in PBS (pH=7.4) containing the primary antibodies: mouse anti-GFAP (G3893 1:400), rabbit anti-GFAP (Sigma #G9269 1:400), mouse anti-S1000 (Sigma #S2532 1:400), rabbit anti-Iba1 (Wako #019-19741, 1:500), rabbit anti-ChAT (Choline acetyltransferase) (Millipore, #AB143, 1:300), mouse anti-CD68 (Abcam, #ab31630, 1:300), rabbit anti-CD34 (Abcam, #ab81289, 1:200), rabbit anti-Ki67 (Abcam, #ab66155, 1:300), rabbit anti-NF-κB-p65 (Abcam, #ab16502, 1:200), rabbit anti-Ki67 (Abcam, #ab16667, 1:200), rabbit anti-ubiquitin (Millipore, #05-1307, 1:200), mouse anti-nitrotyrosine (Millipore, #05233, 1:200), anti-VGlut-1 (SySy, #135-303, 1:300), anti-synaptophysin (Abcam, #206870, 1:300), anti-0111-Tubulin (Millipore, #MAB1637, 1:200).

After washing, sections were incubated in 1:1000-diluted secondary antibodies conjugated to Alexa Fluor 488, Alexa Fluor 546 and/or Alexa Fluor 633 (1:1000, Invitrogen). NeuroTrace 530/615 red fluorescent Nissl stain (Thermo Fisher Scientific) was also used for visualizing neurons.

Immunostaining with rabbit anti-NF-κB-p65, mouse anti-Iba1, rabbit anti-Ki67, mouse anti-CD68, and rabbit anti-ubiquitin required free-floating citrate buffer (sodium citrate 10 mM, pH 6) antigen retrieval at 95° C. for 5 minutes. NeuroTrace 530/615 red fluorescent Nissl stain (Thermo Fisher Scientific, #B34650) was also used for visualizing neurons. After incubation with primary antibodies, free-floating slices were washed with PBS 3 times for 10 min, incubated for 2 h at room temperature with the following secondary antibodies: 1:500 goat anti-rabbit-AlexaFluor 488 (Thermo Fisher Scientific, #A21052), 1:500 goat anti-mouse-AlexaFluor 546 (Thermo Fisher Scientific, #A11035), 1:500 goat anti-mouse-AlexaFluor 633 (Thermo Fisher Scientific, #A21052), 1:500 Streptavidin-AlexaFluor 405 or AlexaFluor 633 (Thermo Fisher Scientific, #S21375), washed with PBS 3 times for 5 minutes and mounted in DPX mounting medium (Sigma, #06522-100ML).

Antibodies were detected by confocal microscopy using a confocal ZEISS LSM 800.

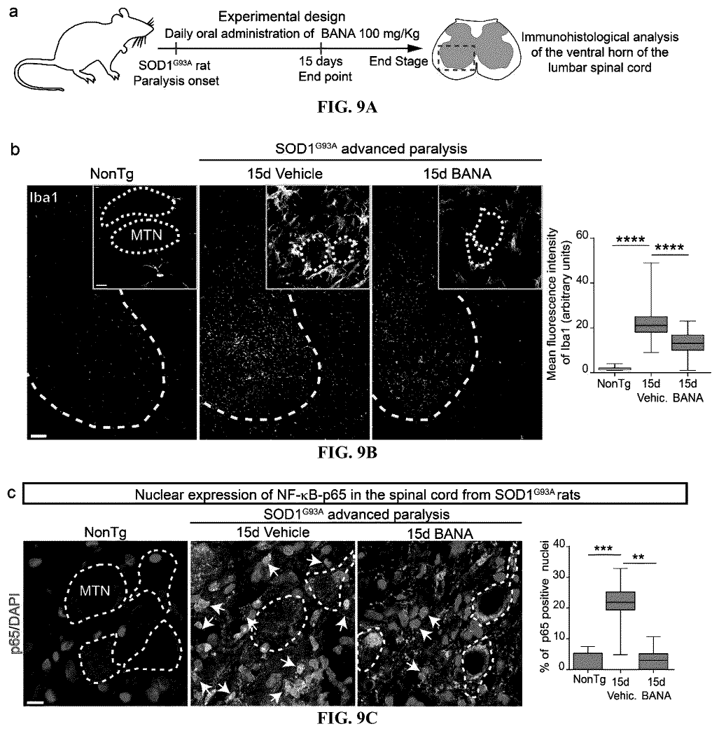

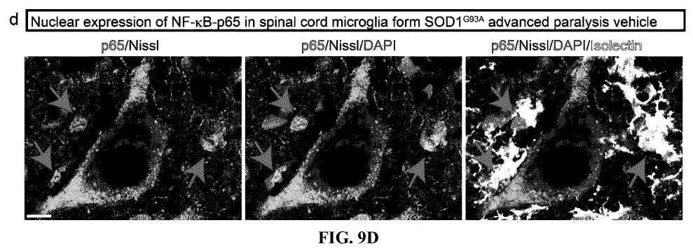

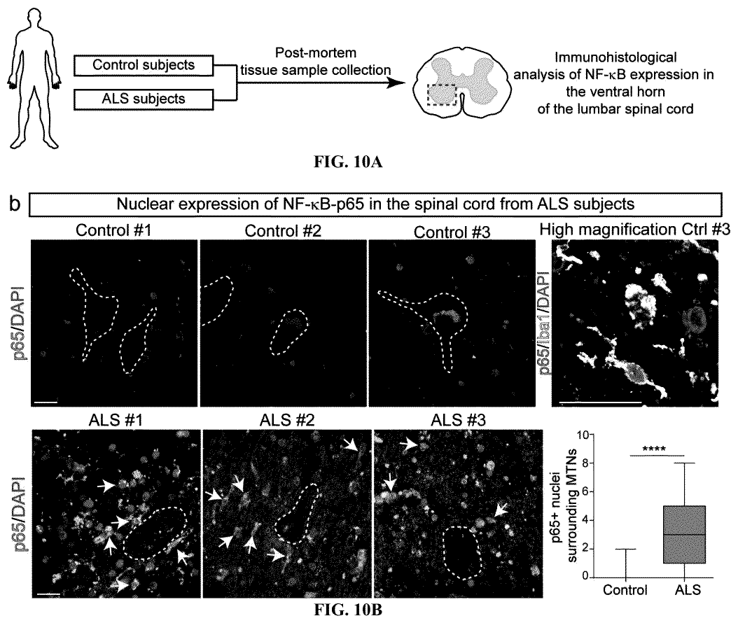

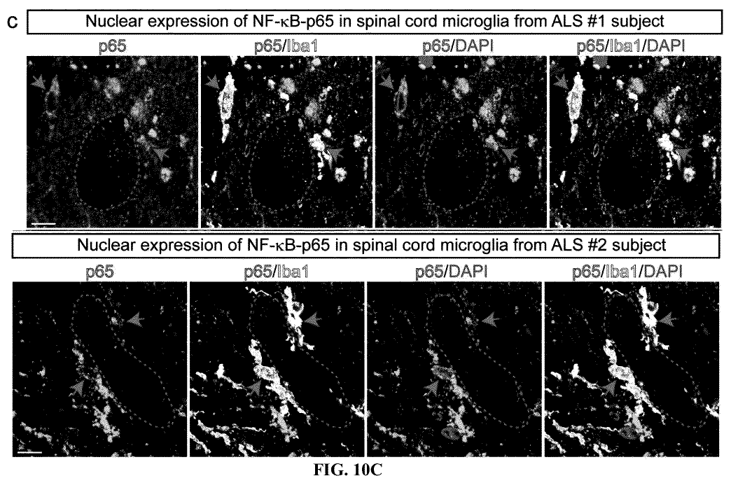

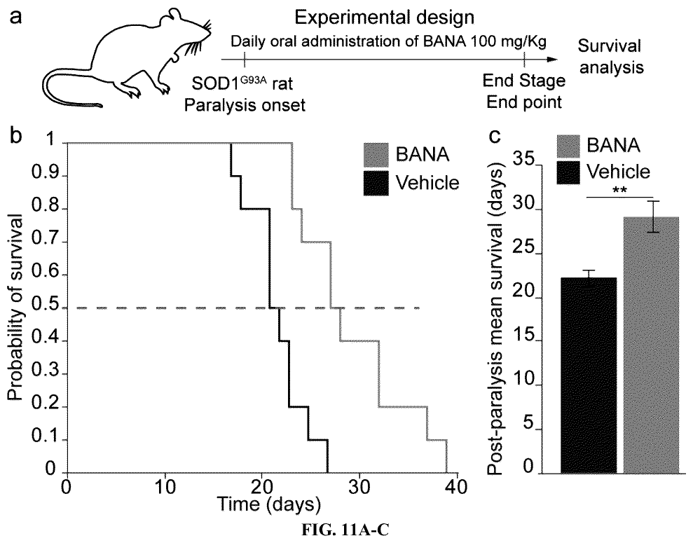

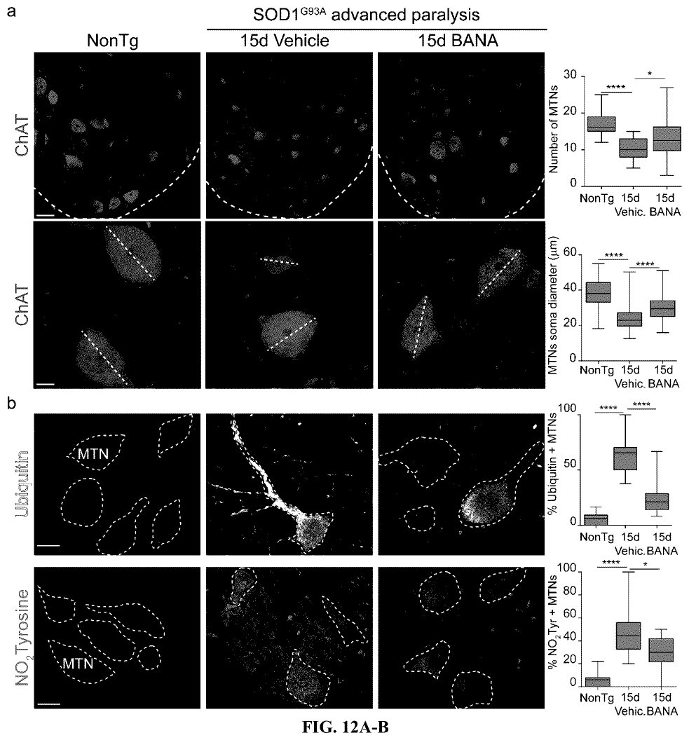

The inventors assessed the effect of BANA on markers of neuroinflammation in the degenerating spinal cords. SOD1^{G93A} rats were treated daily with BANA (100 mg/kg) or vehicle at the onset of motor symptoms. After 15 days of treatment, rats were euthanized and spinal cords were dissected for immunohistological analysis. Compared to vehicle-treated rats, post-paralysis treatment with BANA significantly reduced microgliosis in the ventral horn of the spinal cord as assessed by Iba1-, CD68- and CD34-positive microglial cells (FIG. 9B and FIG. 5A).

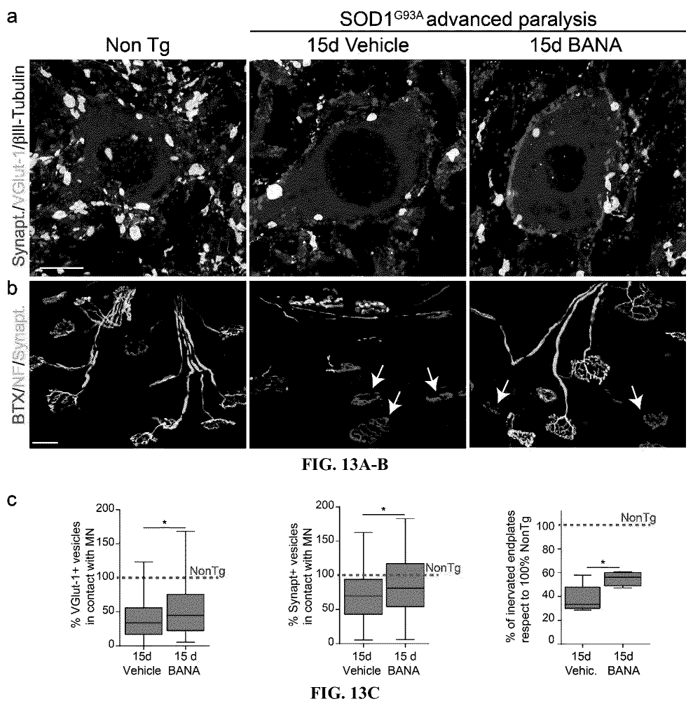

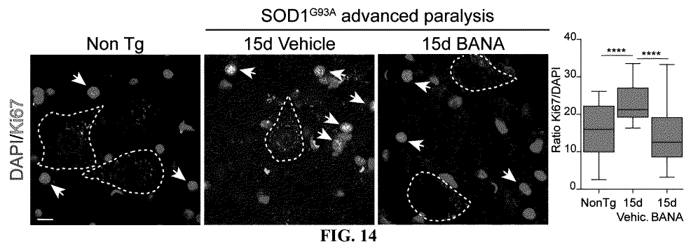

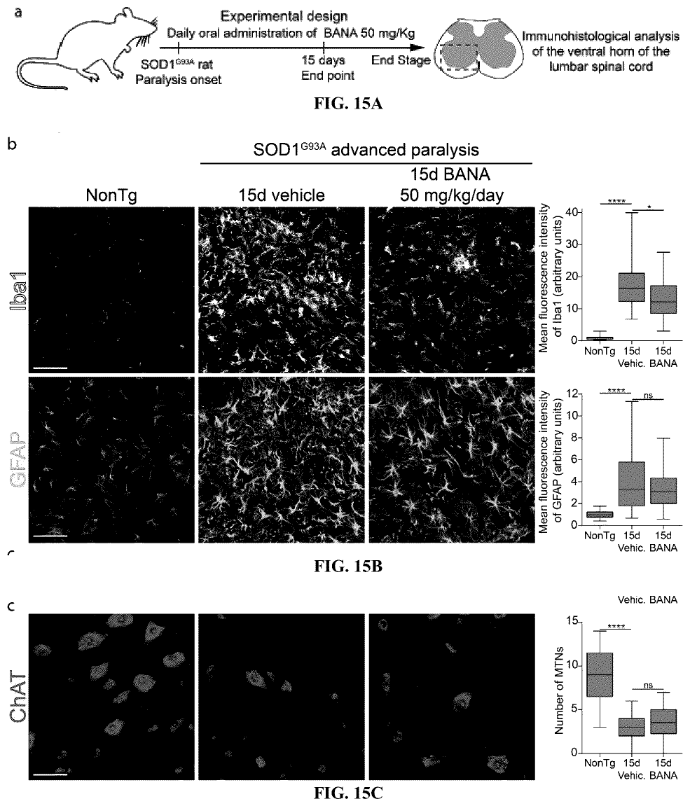

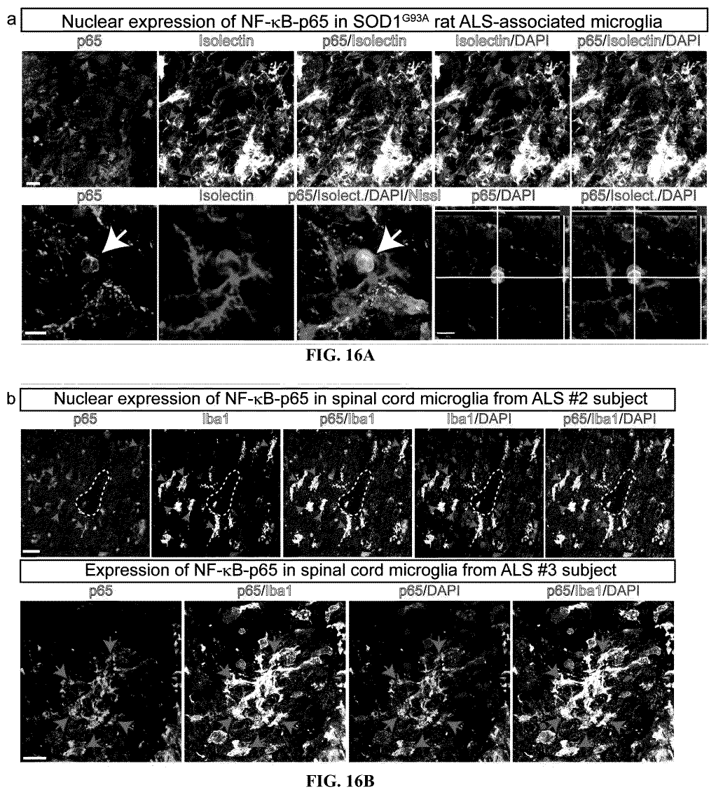

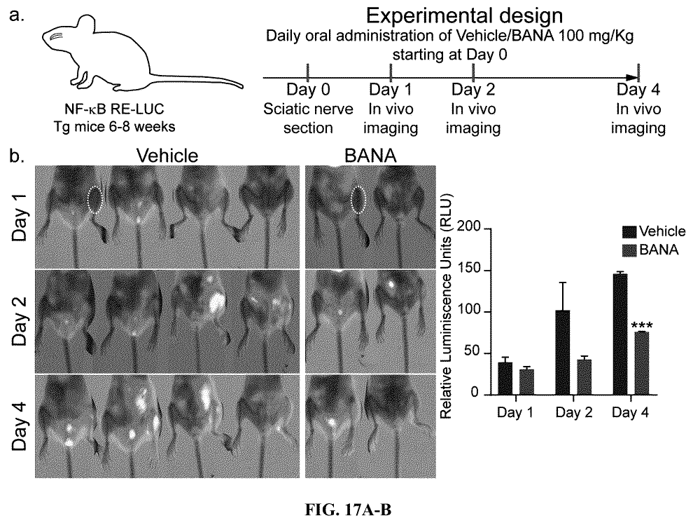

In addition, post-paralysis treatment with BANA significantly reduced astrocytosis assessed by GFAP staining as well as the emergence of aberrant glial cells characterized by the double staining GFAP/S100β in the surroundings of motor neurons (FIG. 13). Treatment with BANA also reduced cell proliferation in the ventral horn as assessed by Ki67-positive nuclei staining (FIG. 14). In comparison, lower doses of BANA (50 mg/kg/day) significantly reduced microgliosis but failed to reduce astrogliosis (FIG. 15A-15C). SOD1^{G93A} female rats were treated with 50 mg/kg/day of BANA or vehicle immediately after the first signs of paralysis onset of one hind limb and continue for 15 days. Immunohistological analysis of the lumbar spinal cord of non-transgenic and vehicle- and BANA-treated SOD1^{G93A} symptomatic rats were performed, the representative confocal microscopy images of which are shown in FIG. 15B. The confocal images show the Iba1 microglia (upper panels) and GFAP astrocytes (lower panels) in the ventral horn of lumbar spinal cord of non-transgenic, vehicle- and BANA-treated SOD1^{G93A} rats. The graphs to the right of the confocal microscopy images show quantitative analysis of microgliosis and astrogliosis in the ventral spinal cord. Data are expressed as mean±SEM; data were analyzed by the Kruskal-Wallis test followed by Dunn's multiple comparisons test, ****p<0.0001, *p=0.0104. n=4 animals per condition. Scale bars=50 μm. FIG. 15C provides representative confocal images of ChAT-positive motor neurons in the ventral horn of lumbar spinal cord of non-transgenic, vehicle- and BANA-treated SOD1^{G93A} rats. The graph to the right of the images represents the quantitative analysis of the number of motor neurons in the ventral horn among conditions. All data are expressed as mean±SEM; data were analyzed by the Kruskal-Wallis test followed by Dunn's multiple comparisons test, ****p<0.0001. n=4 animals per condition. Scale bar=50 μm.

The data provided in FIGS. 15B and 15C shows that treatment with 50 mg/kg/day of BANA reduced microgliosis but not astrogliosis and motor neuron loss.

### Example 9: Analysis of Gliosis in the Ventral Horn of the Lumbar Spinal Cord

Experiments to assess the effect of BANA on markers of neuroinflammation in the degenerating spinal cords were conducted in rats. SOD1^{G93A} rats were treated with BANA (100 mg/kg/day) or vehicle at the onset of symptoms. After 15 days of treatment rats were euthanized and spinal cord was removed for histological analysis.

Microgliosis and astrogliosis were assessed by counting the expression intensity for the different markers in gray matter from the lumbar cord of non-transgenic, SOD1^{G93A} onset or symptomatic rats that were treated with either vehicle or BANA. The number of aberrant glial cells co-expressing the astrocytic markers GFAP and S1000 was assessed by counting the respective positive cells for both markers in gray matter from the lumbar cord of non-transgenic, SOD1^{G93A} onset or symptomatic rats that were treated with either vehicle or BANA. The analysis was performed in at least 10 histological sections per animal (three different rats for each condition) using the ImageJ software. Statistical studies were performed using statistical tools of GraphPad Prism 7 software. Descriptive statistics were used for each group, and Kruskal-Wallis analysis, followed by Dunn's comparison test, was used among groups. All results are presented as mean±SEM, with p<0.05 considered significant.

Compared to vehicle-treated rats, post-paralysis treatment with BANA significantly reduced microgliosis in the ventral horn of spinal cord as assessed by Iba1+ and CD68+ cells (FIG. 5A). BANA also significantly reduced CD34+ cells (FIG. 5A) that are associated with degenerating motor neurons, and decreased the proliferation of cells in the ventral horn of spinal cord as estimated by the proliferation marker Ki67 (FIG. 5B).

These results show that BANA administration after paralysis onset reduces microgliosis and in SOD1^{G93A} rats.

### Example 9: Motor Neuron Analysis

One of the main features of ALS in both human patients and animal models is motor neuron loss, thus the effect of BANA on motor neuron size and number was characterized. To further investigate the protective effect of BANA on motor neuron pathology in SOD1^{G93A} symptomatic rats, experiments were conducted to assess the number of synaptic terminals contacting motor neuron cell bodies in the ventral spinal cord as well as the denervation of neuromuscular junctions.

The number of motor neurons expressing ChAT was assessed by counting the positive cells in the gray matter of the lumbar spinal cord of non-transgenic compared with symptomatic SOD1^{G93A} onset, vehicle- and BANA treated rats. Motor neuron counting was assessed by counting ChAT positive cells in the ventral horn on eight 30 μm sections taken 100 μm apart. The longest axis (length) of each soma was taken into consideration to quantify the mean size of motor neuron soma. The analysis was performed manually in at least 10 histological sections per animal (four different rats for each condition) using the cell counter tool of the ImageJ software. Statistical studies were performed using statistical tools of the GraphPad Prism 7 software. Descriptive statistics were used for each group, and Kruskal-Wallis analysis followed by Dunn's comparison test was used among groups. Results are presented as mean±SEM, with p<0.05 considered significant.

In other experiments, the number of synaptic vesicles contacting motor neuron cell bodies was assessed by immunohistochemistry by counting VGlut-1-positive and Synaptophysin-positive puncta in Rexed laminae VII and IX. Synaptic terminals were quantified in at least 20 motor neurons per animal (4 different rats per condition) using the Analyze Particles tool of ImageJ software.

Post-paralysis treatment with 100 mg/kg/day of BANA significantly prevented the loss of VGlut-1-positive and Synaptophysin-positive synaptic terminals as compared with vehicle-treated rats (FIG. 13A-C). FIG. 13A shows representative confocal images showing 011-Tubulin-positive neurons (blue) in the ventral horn of lumbar spinal cord co-stained with synaptic vesicle markers VGlut-1 (green) and synaptophysin (red). Scale bar=10 μm. FIG. 13B provides NMJs denervation analysis in whole mounted EDL muscles. The panels in FIG. 13B show representative confocal images used to assess the innervation pattern of NMJs in different experimental conditions: a-bungarotoxin-FITC (red) staining was used to analyze motor endplates; synaptophysin-AlexFluor 555 and heavy chain of neurofilaments-AlexaFluor 555 (green) were used to visualize the motor axon branches and presynaptic terminals. The white arrows in FIG. 13B indicate typical denervated motor endplates. Scale bar=50 μm. FIG. 13C provides graphs representing a quantitative analysis of synaptic vesicles in contact with motor neuron cell bodies (FIG. 13C, left and middle graphs, respectively), and NMJ occupancy defined as the overlapping of neurofilament/synaptophysin and αBTX staining (FIG. 13C, far-right graph), expressed as percent respect the non-transgenic condition. All data are expressed as mean±SEM; data were analyzed by Mann-Whitney comparisons test or unpaired t-test between vehicle and BANA groups, *p=0.0474 (VGlut-1), *p=0.0447 (synaptophysin) *p=0.0324 (NMJ). n=4 animals per condition.

Further, representative confocal microscopy images (FIG. 14) show immunohistochemistry analysis of cell proliferation by nuclear Ki67 staining (green) in the surroundings of motor neurons (dotted white lines). The graph on the right of FIG. 14 shows the quantitative analysis of the ratio Ki67/DAPI (green/red) in each condition. BANA significantly reduced cell proliferation when compared with the vehicle-treated group. All quantitative data are expressed as mean±SEM; data were analyzed by Ordinary one-way ANOVA followed by Tukey's multiple comparisons test, **p=0.0013 ****p<0,0001. n=4 animals per condition. Scale bars=15 μm.

The data shows that BANA exerts a protective effect on motor neurons when administrated after paralysis onset.

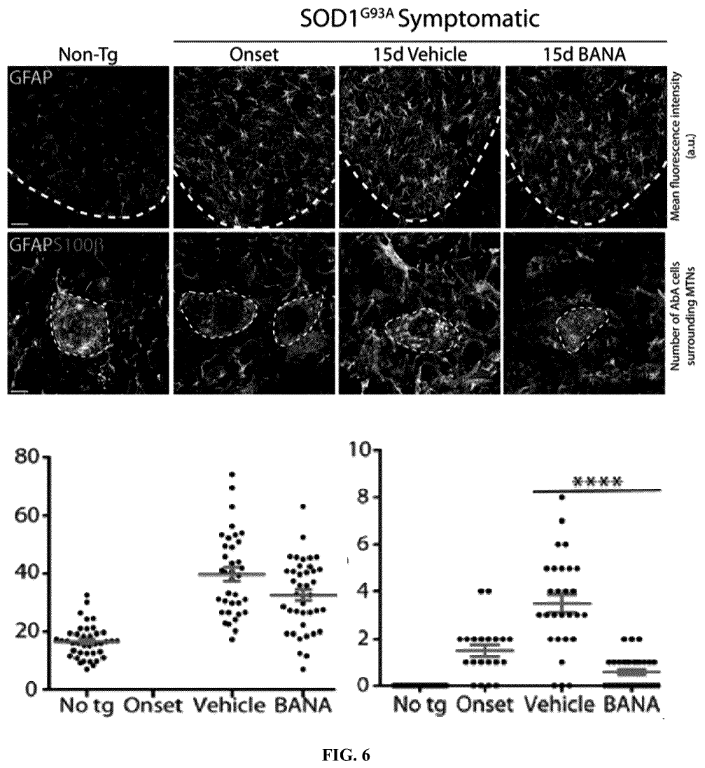

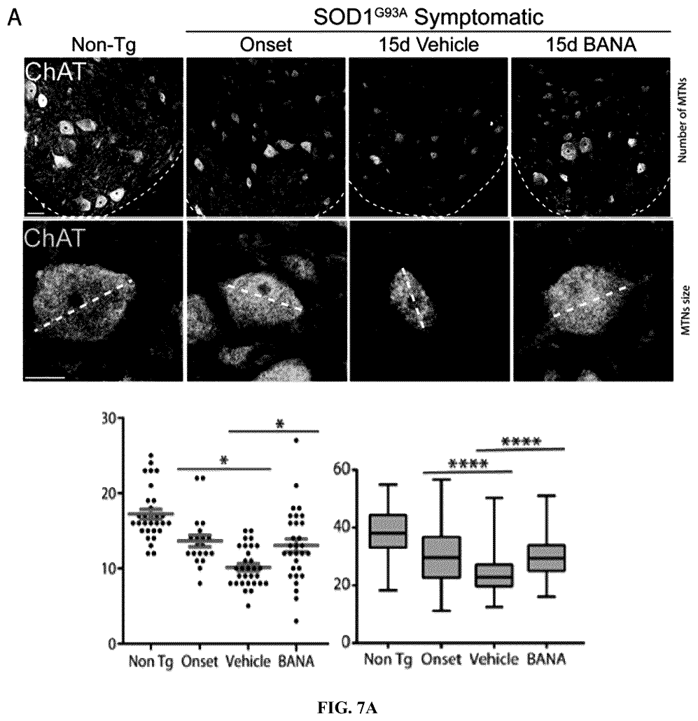

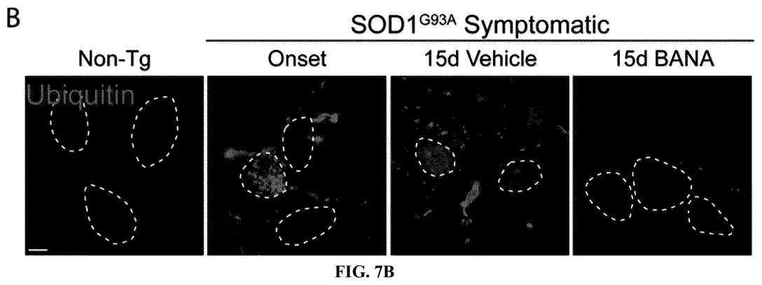

As further shown in FIG. 7A, at the time of paralysis onset the number of ChAT+ cells was reduced by 20%. BANA administration immediately after paralysis onset prevented motor neuron loss maintaining the number of motor neurons as in disease onset. In contrast, the number of ChAT+ neurons in those animals that did not receive BANA was reduced by 60% compared to non-transgenic. BANA also ameliorated the decreased motor neuron size observed during paralysis progression (FIG. 7A) as compared to vehicle-treated rats. BANA treatment also prevented the increased ubiquitin accumulation in damaged motor neurons observed during paralysis progression (FIG. 7B) as compared to vehicle-treated rats, suggesting a neuroprotective effect on motor neuron degeneration. In accordance, BANA treatment significantly reduced motor endplate denervation in the EDL muscle as compared to vehicle-treated rats (FIG. 6).

### Example 10: Immunohistochemistry of Whole Mounted Muscle and Neuromuscular Junction Innervation Analysis

Extensor digitorium longus (EDL) muscles from the NonTg and SOD1^{G93A} hind limbs were dissected. Then, tissues were blocked for 2 h at room temperature (BSA 5% v/v, Triton X-100 0.8% v/v in PBS pH=7.4), incubated with primary antibodies or fluorescent probes at 4° C. overnight: axon and post-synaptic plates were carried out using 1:1000 rabbit anti-heavy chain neurofilament-Alexa Fluor 555 (Millipore, #MAB5256A5) and fluorescently labeled a-bungarotoxin-FITC [αBTX, (Thermo Fisher Scientific, B13422)], axon presynaptic terminals were labeled with 1:300 rabbit anti-synaptophysin-Alexa Fluor 555 (Abcam, #ab206870). After washing 4 times with PBS, whole-mount muscles were mounted using DPX. Structural changes of the neuromuscular junction were scored using maximum-intensity projections of images acquired from whole-mounted muscles. Neuromuscular junction innervation analysis was performed taking into consideration those postsynaptic motor endplates occupied by a presynaptic axon terminal, where full innervation was defined as at least 80% of overlapping between pre- and postsynaptic. An average of 100 neuromuscular junctions per animal was analyzed using the ImageJ software.

### Example 11: BANA Administration after Paralysis Onset Reduces Astrocytosis and the Aberrant Glia and in SOD1^{G93A} Rats

Aberrant glial cells emerge after paralysis onset and exert toxic effect on motor neurons. Chronic treatment with BANA reduced the number of these cells in the ventral horn of spinal cord. As compared with vehicle-treated rats, BANA significantly reduced astrocytosis as assessed by GFAP staining as well as the emergence of aberrant glial cells characterized by the double staining GFAP/S100β in the surroundings of motor neurons (FIG. 6).

Treatment with BANA also reduced cell proliferation in the ventral horn as assessed by Ki67-positive nuclei staining (FIG. 14). Representative confocal images in FIG. 14 show an immunohistochemistry analysis of cell proliferation by nuclear Ki67 staining (green) in the surroundings of motor neurons (dotted white lines). The graph at right of FIG. 14 shows the quantitative analysis of the ratio Ki67/DAPI (green/red) in each condition. BANA significantly reduced cell proliferation when compared with the vehicle-treated group. All quantitative data are expressed as mean±SEM; data were analyzed by Ordinary one-way ANOVA followed by Tukey's multiple comparisons test, **p=0.0013 ****p<0,0001. n=4 animals per condition. Scale bars=15 μm.

In comparison, lower doses of BANA (50 mg/kg/day) significantly reduced microgliosis but failed to reduce astrogliosis (FIGS. 15A-15C). The experimental design shown in FIG. 15A describes how SOD1^{G93A} female rats were treated with 50 mg/kg/day of BANA or vehicle immediately after the first signs of paralysis onset of one hind limb and continue for 15 days. Immunohistological analysis of the lumbar spinal cord of non-transgenic and vehicle- and BANA-treated SOD1^{G93A} symptomatic rats were then performed. Representative confocal images (FIG. 15B) show Iba1 microglia (upper panels) and GFAP astrocytes (lower panels) in the ventral horn of lumbar spinal cord of non-transgenic, vehicle- and BANA-treated SOD1^{G93A} rats. The graphs to the right in FIG. 15B provide a quantitative analysis of microgliosis and astrogliosis in the ventral spinal cord. Data are expressed as mean±SEM; data were analyzed by the Kruskal-Wallis test followed by Dunn's multiple comparisons test, ****p<0.0001, *p=0.0104. n=4 animals per condition. Scale bars=50 μm. FIG. 15C shows representative confocal images of ChAT-positive motor neurons in the ventral horn of lumbar spinal cord of non-transgenic, vehicle- and BANA-treated SOD1^{G93A} rats. The graph at the right of FIG. 15C represents a quantitative analysis of the number of motor neurons in the ventral horn among conditions. All data are expressed as mean±SEM; data were analyzed by the Kruskal-Wallis test followed by Dunn's multiple comparisons test, ****p<0.0001. n=4 animals per condition. Scale bar=50 μm.

### Example 12: BANA Inhibits NF-κB Activation in NF-κB-RE-Luc Transgenic Mice

FIG. 8D shows confocal images of CD11b-positive SOD1^{G93A} primary microglia (grey) treated with BANA, BA or vehicle, before LPS stimulation. BANA inhibits LPS-induced NF-κB-p65 nuclear translocation in SOD1^{G93A} microglia. Nuclear NF-κB-p65 colocalizing with DAPI nuclear staining is denoted in yellow. The graph in FIG. 8E shows the quantitative analysis of the ratio nuclear NF-κB-p65/DAPI among experimental conditions. All quantitative data are expressed as mean±SEM; data were analyzed by Ordinary one-way ANOVA followed by Tukey's multiple comparisons test, ****p<0,0001, 3 replicates of 4 independent experiments. Scale bar=20 μm. FIG. 8F shows an analysis by RT-qPCR of mRNA levels of NF-κB-associated proinflammatory cytokines CCL2, IL-6, IL-1β, and TNFα following LPS-stimulation in SOD1^{G93A} primary microglia. BANA prevents NF-κB-mediated gene transcription induced by LPS. Data are expressed as mean±SEM; data were analyzed by Ordinary one-way ANOVA followed by Tukey's multiple comparisons test, *p=0.0395 (CCL2) *p=0.0409 (IL-6) **p=0.0049 (IL-10) *p=0.0292 **p=0.0063 (TNFα). 2 replicates of 3 independent experiments.

The data show that BANA was particularly active to target NF-κB in microglia cultures including those isolated from the symptomatic SOD1^{G93A} rat spinal cord. SOD1^{G93A} microglia can exhibit an aberrant phenotype with increased proliferation and neurotoxic potential, suggesting they fuel local inflammation and exert a deleterious influence on neuronal survival. In ALS models, the classical NF-κB pathway is related to persistent microglial activation with accelerated disease progression [13, 42]. Here, the inventors identified a specific subset of microglia displaying NF-κB-p65 nuclear translocation localized in the close surrounding of spinal motor neurons. Such microglia appeared to functionally interact with motor neurons in autopsied spinal cords from sporadic ALS patients and also in symptomatic SOD1^{G93A} rats, but not in respective controls. These results are in accordance with previous reports showing 20% of spinal cord microglia displaying nuclear NF-κB-p65 colocalized with TDP43 in sporadic ALS subjects. The data strongly suggest a clear causal association of nuclear NF-κB in microglia with progressive motor neuron damage and neuromuscular junction loss. Notably, post-paralysis treatment with BANA for 15 days resulted in a significant reduction of perineuronal microglia bearing NF-κB-p65-positive nuclei, suggesting the potential of BANA to target this particular subset of microglia.

The effects of BANA on LPS- or TNFα-induced NF-κB activation were also assayed in cell cultures of BV2 microglial cell line and primary adult microglia isolated from symptomatic SOD1^{G93A} rats, and HT-29 NF-κB reporter cell line, respectively. As shown in FIGS. 8C, 8D, and 8G, BANA (10-30 μM) potently prevented NF-κB activation as assessed by either LPS-induced NF-κB-p65 nuclear translocation in BV2 cells and SOD1^{G93A} microglia or TNFα-induced NF-κB-p65 nuclear translocation in HT-29 NF-κB reporter cell line. In addition, BANA prevented LPS-induced upregulation of NF-κB-dependent transcriptional activity of MCP1, IL-6, IL-1β, and TNFα assessed by RT-qPCR in adult microglia isolated from symptomatic SOD1^{G93A} rats (FIG. 8F). The benzoic acid scaffold and dimethyl fumarate were devoid of inhibitory effect in NF-κB pathways in these experimental settings. The LC₅₀ values assessed in cell cultures were 50 μM, 30 μM, and 80 μM, for BV2, SOD1^{G93A} adult microglia, and HT-29, respectively.

### Example 13: Effect of BANA on NF-kB Activation after Sciatic Nerve Section in Mice

For NF-kB in vivo imaging studies, the NF-kB-RE-Luc random transgenic mouse model (Taconic, BALB/c-Tg(Rela-luc)31Xen) aged 6-8 weeks was used. These animals carry a transgene containing 6 NF-kB-responsive elements (RE) from the CMVα (immediate early) promoter placed upstream of a basal SV40 promoter, and a modified firefly luciferase cDNA (Promega pGL3). Animals were randomized divided into two groups and orally administrated with 100 mg/kg of BANA or Vehicle (phosphate buffer) starting immediately before sciatic nerve section in the right hindlimb and continuing for 4 days. At day 1, 2 and 4 post-surgery, differences in luciferase activity were compared between groups. To accomplish that, 150 mg/kg of the substrate D-luciferin (#K9918PE, XenoLight) dissolved in PBS, pH 7.4, was injected intraperitoneally to each mouse. Animals were anesthetized with isoflurane (3.0% induction, 2.5% maintenance), placed in ventral position in the light-tight imaging chamber and imaged 10 minutes post luciferin injection using bioluminescence and X-ray modes (5 seconds acquisition, performed in Preclinical In-Vivo Xtreme II Optical/X-ray imaging system, Bruker, USA). Changes in NF-kB activity were quantified after comparing the differences of luminescence in the right hindlimb between groups, using ImageJ software.

### Example 14: Human Tissue Collection

The collection of postmortem human ALS and control samples was approved by the University of Alabama, Birmingham (UAB) Institutional Review Board (Approved IRB Protocol: X091222037 to Dr. Peter H. King). All ALS patients were cared for at UAB and so detailed clinical records were available. Control samples were age-matched and were harvested from patients who expired from non-neurological causes. The average collection time after death was less than 10 h. All tissues were harvested by PHK and YS at the time of autopsy and preserved within 30 minutes.

Spinal cord sections (10 m) in paraffin were sliced using a microtome. Following deparaffinization, slices were blocked and permeabilized in BSA 5% (v/v)/Triton X-100 0.5% (v/v) for 2 h at room temperature. The above-described primary antibodies were incubated in BSA 1% (v/v)/Triton X-100 0.5% (v/v) in PBS pH=7.4 at 4° C. overnight. After washing, secondary antibodies were incubated for 3 h at room temperature. After PBS washing, Mowiol medium (Sigma, St. Louis, MO, USA) was used for mounting. Only ventral lumbar spinal cord sections were analyzed. Motor neuron somas were identified in the ventral spinal cord by typical morphology and nuclei.

Fluorescence imaging was performed with a laser scanning Zeiss LSM 800 or LSM 880 confocal microscope with either a 25× (1.2 numerical aperture) objective or 63× (1.3 numerical aperture) oil immersion objective using Zeiss Zen Black software. Maximum intensity projections of optical sections were created with Zeiss Zen software. Maximum intensity projections of optical sections, as well as 3D reconstructions, were created with Zeiss Zen software.

### Discussion of Experimental Results

In some embodiments, the present invention concerns the synthesis, biological, and therapeutic effects of (E)-4-(2-nitrovinyl)benzoic acid (BANA) in cellular and animal models of ALS. Experiments demonstrated BANA's potential to downregulate deleterious microglia activation through inhibition of NF-κB as compared with dimethyl fumarate, a well-known electrophilic neuroprotective marketed drug [29]. BANA reduced neuroinflammation and slowed disease progression in the transgenic SOD1^{G93A} rat.

SOD1^{G93A} rats receiving post-paralysis treatment with BANA not only had prolonged survival and decreased nuclear NF-κB-p65-positive microglia but also exhibited healthier motor neurons as assessed by reduction of neurons displaying nitrotyrosine and ubiquitin staining. Accumulation of ubiquitin immunoreactivity denotes ER stress and proteinopathy while nitrotyrosine staining is a marker of oxidative and nitrative stress preceding apoptosis in motor neurons. BANA treatment also preserved the reduction of the number, size and synaptic inputs of spinal motor neurons as well as the number of innervated neuromuscular junctions in the EDL muscle, further confirming the neuroprotective effect of BANA in ALS SOD1^{G93A} rats.

While not being bound by a particular theory, it is unlikely that BANA's neuroprotective effect in SOD1^{G93A} rats is solely due to its specific effect on NF-κB-activated microglia. BANA could target other cytoprotective signaling pathways in addition to NF-κB, including Nrf2-ARE and/or This specification discloses one or more embodiments that incorporate the features of this invention. The disclosed embodiment(s) merely exemplify the invention. The scope of the invention is not limited to the disclosed embodiment(s). The invention is defined by the claims appended hereto.

After oral administration, BANA reaches a low micromolar concentration in plasma. The inventors infer that BANA can be a substrate of monocarboxylic acid type-1 transporters, which are known to transport benzoic acid and salicylic acid to the CNS. Evidence also indicates significant alterations in the blood-spinal cord barrier in ALS, including endothelial cells and pericyte degeneration, capillary leakage, downregulation of tight junction proteins, and microhemorrhages, which likely play a pathogenic mechanism aggravating motor neuron damage. Because restoring blood-brain-barrier integrity retards the disease process in ALS animal models, BANA may also protect vascular pathology in ALS through modulation of NF-κB signaling in endothelial cells and pericytes.

In sum, the data provided herein demonstrate the neuroprotective effect of the nitroalkene benzoic acid derivative BANA in a model of inherited ALS exerting a disease-modifying effect when administered after paralysis onset. While the inhibitory effects of BANA on NF-κB activation is systemic and possibly involves multifaceted cell types, strikingly, the present data identifies a subset of NF-κB-positive ALS-associated microglia surrounding spinal motor neurons as pathogenic-relevant targets of the drug. This data has shown for the first time the emergence of such perineuronal NF-κB-positive microglia in autopsy samples from sporadic ALS cases, showing that the pathological microglia-motor neuron crosstalk in ALS can be pharmacology targeted by a nitroalkene derivative such as BANA.

### CONCLUSION

The examples described herein illustrate possible embodiments of the present invention. While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

### REFERENCES

1. Brown, R H and A. Al-Chalabi, Amyotrophic Lateral Sclerosis. N Engl J Med, 2017. 377(2): p. 162-172.
2. Miller R G, Mitchell J D, Lyon M, Moore D H. Riluzole for amyotrophic lateral sclerosis (ALS)/motor neuron disease (MND). Amyotrophic lateral sclerosis and other motor neuron disorders: official publication of the World Federation of Neurology, Research Group on Motor Neuron Diseases. 2003; 4(3):191-206.
3. Yohrling, G. J. T., et al., Analysis of cellular, transgenic and human models of Huntington's disease reveals tyrosine hydroxylase alterations and substantia nigra neuropathology. Brain Res Mol Brain Res, 2003. 119(1): p. 28-36.
4. Vargas, M. R., et al., Stimulation of nerve growth factor expression in astrocytes by peroxynitrite. In Vivo, 2004. 18(3): p. 269-74.
5. Ilieva, H., M. Polymenidou, and D. W. Cleveland, Non-cell autonomous toxicity in neurodegenerative disorders: ALS and beyond. J Cell Biol, 2009. 187(6): p. 761-72.
6. Maragakis, N. J. and J. D. Rothstein, Mechanisms of Disease: astrocytes in neurodegenerative disease. Nat Clin Pract Neurol, 2006. 2(12): p. 679-89.
7. Diaz-Amarilla, P., et al., Phenotypically aberrant astrocytes that promote motoneuron damage in a model of inherited amyotrophic lateral sclerosis. Proc Natl Acad Sci USA, 2011. 108(44): p. 18126-31. doi: 10.1073/pnas.1110689108.
8. Trias, E., et al., Significance of aberrant glial cell phenotypes in pathophysiology of amyotrophic lateral sclerosis. Neurosci Lett, 2017. 636: p. 27-31.
9. Trias, E., et al., Phenotypic transition of microglia into astrocyte-like cells associated with disease onset in a model of inherited ALS. Front Cell Neurosci, 2013. 7: p. 274. doi: 10.3389/fncel.2013.00274.
10. McCauley, M. E. and R. H. Baloh, Inflammation in ALS/FTD pathogenesis. Acta Neuropathol, 2019. 137(5): p. 715-730. doi: 10.1007/s00401-018-1933-9.
11. Wijesekera, L. C. and P. N. Leigh, Amyotrophic lateral sclerosis. Orphanet J Rare Dis, 2009. 4: p. 3.
12. Frakes, A. E., et al., Microglia induce motor neuron death via the classical NF-kappaB pathway in amyotrophic lateral sclerosis. Neuron, 2014. 81(5): p. 1009-1023. doi: 10.1016/j.neuron.2014.01.013.
13. Trias E, Ibarburu S, Barreto-Nunez R, Babdor J, Maciel T T, Guillo M, et al. et al., Post-paralysis tyrosine kinase inhibition with masitinib abrogates neuroinflammation and slows disease progression in inherited amyotrophic lateral sclerosis. J Neuroinflammation, 2016. 13(1): p. 177. doi: 10.1186/s12974-016-0620-9.
14. Geloso, M. C., et al., The Dual Role of Microglia in ALS: Mechanisms and Therapeutic Approaches. Front Aging Neurosci, 2017. 9: p. 242.
15. Montes Diaz, G., et al., Dimethyl fumarate induces a persistent change in the composition of the innate and adaptive immune system in multiple sclerosis patients. Sci Rep, 2018. 8(1): p. 8194.
16. Staun-Ram, E., et al., Dimethyl fumarate as a first- vs second-line therapy in MS: Focus on B cells. Neurol Neuroimmunol Neuroinflamm, 2018. 5(6): p. e508.
17. de Paula, C. Z., B. D. Goncalves, and L. B. Vieira, An Overview of Potential Targets for Treating Amyotrophic Lateral Sclerosis and Huntington's Disease. Biomed Res Int, 2015. 2015: p. 198612.
18. Mills, E. A., et al., Emerging Understanding of the Mechanism of Action for Dimethyl Fumarate in the Treatment of Multiple Sclerosis. Front Neurol, 2018. 9: p. 5. doi: 10.3389/fneur.2018.00005
19. Kansanen, E., et al., Electrophilic nitro-fatty acids activate NRF2 by a KEAP1 cysteine 151-independent mechanism. J Biol Chem, 2011. 286(16): p. 14019-27.
20. Kansanen, E., H. K. Jyrkkanen, and A. L. Levonen, Activation of stress signaling pathways by electrophilic oxidized and nitrated lipids. Free Radic Biol Med, 2012. 52(6): p. 973-82.
21. Cui, T., et al., Nitrated fatty acids: Endogenous anti-inflammatory signaling mediators. J Biol Chem, 2006. 281(47): p. 35686-98. doi: 10.1074/jbc.M603357200
22. Villacorta, L., et al., Electrophilic nitro-fatty acids inhibit vascular inflammation by disrupting LPS-dependent TLR4 signalling in lipid rafts. Cardiovasc Res, 2013. 98(1): p. 116-24.
23. Diaz-Amarilla, P., et al., Electrophilic nitro-fatty acids prevent astrocyte-mediated toxicity to motor neurons in a cell model of familial amyotrophic lateral sclerosis via nuclear factor erythroid 2-related factor activation. Free Radic Biol Med, 2016. 95: p. 112-20. doi: 10.1016/j.freeradbiomed.2016.03.013.
24. Schopfer, F. J., et al., Nitrolinoleic acid: an endogenous peroxisome proliferator-activated receptor gamma ligand. Proc Natl Acad Sci USA, 2005. 102(7): p. 2340-5.
25. Kansanen, E., et al., Nrf2-dependent and -independent responses to nitro-fatty acids in human endothelial cells: identification of heat shock response as the major pathway activated by nitro-oleic acid. J Biol Chem, 2009. 284(48): p. 33233-41.
26. Writing, G. and A. L. S. S. G. Edaravone, Safety and efficacy of edaravone in well defined patients with amyotrophic lateral sclerosis: a randomised, double-blind, placebo-controlled trial. Lancet Neurol, 2017. 16(7): p. 505-512. doi: 10.1016/S1474-4422(17)30115-1.
27. Howland D S, Liu J, She Y, Goad B, Maragakis N J, Kim B, et al. Focal loss of the glutamate transporter EAAT2 in a transgenic rat model of SOD1 mutant-mediated amyotrophic lateral sclerosis (ALS). Proceedings of the National Academy of Sciences of the United States of America. 2002; 99(3):1604-9. doi: 10.1073/pnas.032539299.
28. Trias E, Ibarburu S, Barreto-Nunez R, Barbeito L. Significance of aberrant glial cell phenotypes in pathophysiology of amyotrophic lateral sclerosis. Neurosci Lett. 2017; 636:27-31. doi: 10.1016/j.neulet.2016.07.052.
29. Mora J S, Genge A, Chio A, Estol C J, Chaverri D, Hernandez M, et al. Masitinib as an add-on therapy to riluzole in patients with amyotrophic lateral sclerosis: a randomized clinical trial. Amyotroph Lateral Scler Frontotemporal Degener. 2020; 21(1-2):5-14. doi: 10.1080/21678421.2019.1632346.
30. Tak P P, Firestein G S. NF-kappaB: a key role in inflammatory diseases. J Clin Invest. 2001; 107(1):7-11. doi: 10.1172/JCI11830.
31. Kaltschmidt B, Kaltschmidt C. NF-kappaB in the nervous system. Cold Spring Harb Perspect Biol. 2009; 1(3):a001271. doi: 10.1101/cshperspect.a001271.
32. Liu T, Zhang L, Joo D, Sun S C. NF-kappaB signaling in inflammation. Signal transduction and targeted therapy. 2017; 2. doi: 10.1038/sigtrans.2017.23.
33. Dresselhaus E C, Meffert M K. Cellular Specificity of NF-kappaB Function in the Nervous System. Frontiers in immunology. 2019; 10:1043. doi: 10.3389/fimmu.2019.01043.
34. Ju Hwang C, Choi D Y, Park M H, Hong J T. NF-kappaB as a Key Mediator of Brain Inflammation in Alzheimer's Disease. CNS & neurological disorders drug targets. 2019; 18(1):3-10. doi: 10.2174/1871527316666170807130011.
35. Swarup V, Phaneuf D, Dupre N, Petri S, Strong M, Kriz J, et al. Deregulation of TDP-43 in amyotrophic lateral sclerosis triggers nuclear factor kappaB-mediated pathogenic pathways. The Journal of experimental medicine. 2011; 208(12):2429-47. doi: 10.1084/jem.20111313.
36. Sako W, Ito H, Yoshida M, Koizumi H, Kamada M, Fujita K, et al. Nuclear factor kappa B expression in patients with sporadic amyotrophic lateral sclerosis and hereditary amyotrophic lateral sclerosis with optineurin mutations. Clinical neuropathology. 2012; 31(6):418-23. doi: 10.5414/NP300493.
37. Khoo N K H, Li L, Salvatore S R, Schopfer F J, Freeman B A. Electrophilic fatty acid nitroalkenes regulate Nrf2 and NF-kappaB signaling: A medicinal chemistry investigation of structure-function relationships. Sci Rep. 2018; 8(1):2295. doi: 10.1038/s41598-018-20460-8.
38. Rodriguez-Duarte J, Dapueto R, Galliussi G, Turell L, Kamaid A, Khoo N K H, et al. Electrophilic nitroalkene-tocopherol derivatives: synthesis, physicochemical characterization and evaluation of anti-inflammatory signaling responses. Scientific reports. 2018; 8(1):12784. doi: 10.1038/s41598-018-31218-7.
39. Rodriguez-Duarte J, Galliussi G, Dapueto R, Rossello J, Malacrida L, Kamaid A, et al. A novel nitroalkene-alpha-tocopherol analogue inhibits inflammation and ameliorates atherosclerosis in Apo E knockout mice. British journal of pharmacology. 2019; 176(6):757-72. doi: 10.1111/bph.14561.
40. Bridges J W, French M R, Smith R L, Williams R T. The fate of benzoic acid in various species. The Biochemical journal. 1970; 118(1):47-51. doi: 10.1042/bj1180047.
41. Leonard J V, Morris A A. Urea cycle disorders. Seminars in neonatology: S N. 2002; 7(1):27-35. doi: 10.1053/siny.2001.0085.
42. Modi K K, Roy A, Brahmachari S, Rangasamy S B, Pahan K. Cinnamon and Its Metabolite Sodium Benzoate Attenuate the Activation of p21rac and Protect Memory and Learning in an Animal Model of Alzheimer's Disease. PloS one. 2015; 10(6):e0130398. doi: 10.1371/journal.pone.0130398.
43. Khasnavis S, Pahan K. Sodium benzoate, a metabolite of cinnamon and a food additive, upregulates neuroprotective Parkinson disease protein DJ-1 in astrocytes and neurons. Journal of neuroimmune pharmacology: the official journal of the Society on NeuroImmune Pharmacology. 2012; 7(2):424-35. doi: 10.1007/s11481-011-9286-3.
44. Yin M J, Yamamoto Y, Gaynor R B. The anti-inflammatory agents aspirin and salicylate inhibit the activity of I(kappa)B kinase-beta. Nature. 1998; 396(6706):77-80. doi: 10.1038/23948.
45. Grilli M, Pizzi M, Memo M, Spano P. Neuroprotection by aspirin and sodium salicylate through blockade of NF-kappaB activation. Science. 1996; 274(5291):1383-5. doi: 10.1126/science.274.5291.1383.
46. Albrecht P, Bouchachia I, Goebels N, Henke N, Hofstetter H H, Issberner A, et al. Effects of dimethyl fumarate on neuroprotection and immunomodulation. Journal of neuroinflammation. 2012; 9:163. doi: 10.1186/1742-2094-9-163.
47. Miller S A, White J A, Chowdhury R, Gales D N, Tameru B, Tiwari A K, et al. Effects of consumption of whole grape powder on basal NF-kappaB signaling and inflammatory cytokine secretion in a mouse model of inflammation. Journal of nutrition & intermediary metabolism. 2018; 11:1-8. doi: 10.1016/j.jnim.2017.11.002.
48. Mastropietro G, Tiscornia I, Perelmuter K, Astrada S, Bollati-Fogolin M. HT-29 and Caco-2 reporter cell lines for functional studies of nuclear factor kappa B activation. Mediators of inflammation. 2015; 2015:860534. doi: 10.1155/2015/860534.
49. Thomsen G M, Gowing G, Latter J, Chen M, Vit J P, Staggenborg K, et al. Delayed disease onset and extended survival in the SOD1^{G93A} rat model of amyotrophic lateral sclerosis after suppression of mutant SOD1 in the motor cortex. The Journal of neuroscience: the official journal of the Society for Neuroscience. 2014; 34(47):15587-600. doi: 10.1523/JNEUROSCI.2037-14.2014.
50. Trias E, Ibarburu S, Barreto-Nunez R, Varela V, Moura I C, Dubreuil P, et al. Evidence for mast cells contributing to neuromuscular pathology in an inherited model of ALS. JCI Insight. 2017; 2(20). doi: 10.1172/jci.insight.95934.
51. Kovacs M, Trias E, Varela V, Ibarburu S, Beckman J S, Moura I C, et al. CD34 Identifies a Subset of Proliferating Microglial Cells Associated with Degenerating Motor Neurons in ALS. International journal of molecular sciences. 2019; 20(16). doi: 10.3390/ijms20163880.
52. Vucic S, Ryder J, Mekhael L, Rd H, Mathers S, Needham M, et al. Phase 2 randomized placebo controlled double blind study to assess the efficacy and safety of tecfidera in patients with amyotrophic lateral sclerosis (TEALS Study): Study protocol clinical trial (SPIRIT Compliant). Medicine. 2020; 99(6):e18904. doi: 10.1097/M D.0000000000018904.
53. Brahmachari S, Jana A, Pahan K. Sodium benzoate, a metabolite of cinnamon and a food additive, reduces microglial and astroglial inflammatory responses. Journal of immunology. 2009; 183(9):5917-27. doi: 10.4049/jimmunol.0803336.
54. Trias E, Beilby P R, Kovacs M, Ibarburu S, Varela V, Barreto-Nunez R, et al. Emergence of Microglia Bearing Senescence Markers During Paralysis Progression in a Rat Model of Inherited ALS. Frontiers in aging neuroscience. 2019; 11:42. doi: 10.3389/fnagi.2019.00042.
55. Leigh P N, Whitwell H, Garofalo O, Buller J, Swash M, Martin J E, et al. Ubiquitin-immunoreactive intraneuronal inclusions in amyotrophic lateral sclerosis. Morphology, distribution, and specificity. Brain: a journal of neurology. 1991; 114 (Pt 2):775-88.
56. Beal M F, Ferrante R J, Browne S E, Matthews R T, Kowall N W, Brown R H, Jr. Increased 3-nitrotyrosine in both sporadic and familial amyotrophic lateral sclerosis. Annals of neurology. 1997; 42(4):644-54. doi: 10.1002/ana.410420416.
57. Kim M C, Kim S J, Kim D S, Jeon Y D, Park S J, Lee H S, et al. Vanillic acid inhibits inflammatory mediators by suppressing NF-kappaB in lipopolysaccharide-stimulated mouse peritoneal macrophages. Immunopharmacol Immunotoxicol. 2011; 33(3):525-32. doi: 10.3109/08923973.2010.547500.
58. Patel P, Julien J P, Kriz J. Early-stage treatment with Withaferin A reduces levels of misfolded superoxide dismutase 1 and extends lifespan in a mouse model of amyotrophic lateral sclerosis. Neurotherapeutics: the journal of the American Society for Experimental NeuroTherapeutics. 2015; 12(1):217-33. doi: 10.1007/s13311-014-0311-0.
59. Shruthi K, Reddy S S, Chitra P S, Reddy G B. Ubiquitin-proteasome system and E R stress in the brain of diabetic rats. Journal of cellular biochemistry. 2019; 120(4):5962-73. doi: 10.1002/jcb.27884.
60. Dantuma N P, Bott L C. The ubiquitin-proteasome system in neurodegenerative diseases: precipitating factor, yet part of the solution. Frontiers in molecular neuroscience. 2014; 7:70. doi: 10.3389/fnmol.2014.00070.
61. Estevez A G, Spear N, Manuel S M, Radi R, Henderson C E, Barbeito L, et al. Nitric oxide and superoxide contribute to motor neuron apoptosis induced by trophic factor deprivation. The Journal of neuroscience: the official journal of the Society for Neuroscience. 1998; 18(3):923-31.
62. Lipton S A, Rezaie T, Nutter A, Lopez K M, Parker J, Kosaka K, et al. Therapeutic advantage of pro-electrophilic drugs to activate the Nrf2/ARE pathway in Alzheimer's disease models. Cell death & disease. 2016; 7(12):e2499. doi: 10.1038/cddis.2016.389.
63. Schopfer F J, Vitturi D A, Jorkasky D K, Freeman B A. Nitro-fatty acids: New drug candidates for chronic inflammatory and fibrotic diseases. Nitric oxide: biology and chemistry. 2018; 79:31-7. doi: 10.1016/j.niox.2018.06.006.
64. Bright J J, Kanakasabai S, Chearwae W, Chakraborty S. PPAR Regulation of Inflammatory Signaling in CNS Diseases. PPAR research. 2008; 2008:658520. doi: 10.1155/2008/658520.
65. Corona J C, Duchen M R. PPARgamma as a therapeutic target to rescue mitochondrial function in neurological disease. Free radical biology & medicine. 2016; 100:153-63. doi: 10.1016/j.freeradbiomed.2016.06.023.
66. Satoh T, Okamoto S I, Cui J, Watanabe Y, Furuta K, Suzuki M, et al. Activation of the Keap1/Nrf2 pathway for neuroprotection by electrophilic [correction of electrophillic] phase II inducers. Proceedings of the National Academy of Sciences of the United States of America. 2006; 103(3):768-73. doi: 10.1073/pnas.0505723102. 51. Srinivasan M, Lahiri D K. Significance of NF-kappaB as a pivotal therapeutic target in the neurodegenerative pathologies of Alzheimer's disease and multiple sclerosis. Expert opinion on therapeutic targets. 2015; 19(4):471-87. doi: 10.1517/14728222.2014.989834.
67. Bomprezzi R. Dimethyl fumarate in the treatment of relapsing-remitting multiple sclerosis: an overview. Therapeutic advances in neurological disorders. 2015; 8(1):20-30. doi: 10.1177/1756285614564152.
68. Carlstrom K E, Ewing E, Granqvist M, Gyllenberg A, Aeinehband S, Enoksson S L, et al. Therapeutic efficacy of dimethyl fumarate in relapsing-remitting multiple sclerosis associates with ROS pathway in monocytes. Nature communications. 2019; 10(1):3081. doi: 10.1038/s41467-019-11139-3.
69. Kastrati I, Siklos M I, Calderon-Gierszal E L, El-Shennawy L, Georgieva G, Thayer E N, et al. Dimethyl Fumarate Inhibits the Nuclear Factor kappaB Pathway in Breast Cancer Cells by Covalent Modification of p65 Protein. The Journal of biological chemistry. 2016; 291(7):3639-47. doi: 10.1074/jbc.M115.679704.
70. Trias E, King P H, Si Y, Kwon Y, Varela V, Ibarburu S, et al. Mast cells and neutrophils mediate peripheral motor pathway degeneration in ALS. JCI Insight. 2018; 3(19). doi: 10.1172/jci.insight.123249.
71. Graves M C, Fiala M, Dinglasan L A, Liu N Q, Sayre J, Chiappelli F, et al. Inflammation in amyotrophic lateral sclerosis spinal cord and brain is mediated by activated macrophages, mast cells and T cells. Amyotrophic lateral sclerosis and other motor neuron disorders: official publication of the World Federation of Neurology, Research Group on Motor Neuron Diseases. 2004; 5(4):213-9. doi: 10.1080/14660820410020286.
72. Nagano S, Takahashi Y, Yamamoto K, Masutani H, Fujiwara N, Urushitani M, et al. A cysteine residue affects the conformational state and neuronal toxicity of mutant SOD1 in mice: relevance to the pathogenesis of ALS. Hum Mol Genet. 2015; 24(12):3427-39. doi: 10.1093/hmg/ddv093.
73. Williams J R, Trias E, Beilby P R, Lopez N I, Labut E M, Bradford C S, et al. Copper delivery to the CNS by CuATSM effectively treats motor neuron disease in SOD1^{G93A} mice co-expressing the Copper-Chaperone-for-SOD. Neurobiol Dis. 2016; 89:1-9. doi: 10.1016/j.nbd.2016.01.020.
74. Tsuji A. Small molecular drug transfer across the blood-brain barrier via carrier-mediated transport systems. NeuroRx: the journal of the American Society for Experimental NeuroTherapeutics. 2005; 2(1):54-62. doi: 10.1602/neurorx.2.1.54.
75. Garbuzova-Davis S, Sanberg P R. Blood-CNS Barrier Impairment in ALS patients versus an animal model. Front Cell Neurosci. 2014; 8:21. doi: 10.3389/fncel.2014.00021.
76. Winkler E A, Sengillo J D, Sagare A P, Zhao Z, Ma Q, Zuniga E, et al. Blood-spinal cord barrier disruption contributes to early motor-neuron degeneration in ALS-model mice. Proceedings of the National Academy of Sciences of the United States of America. 2014; 111(11):E1035-42. doi: 10.1073/pnas.1401595111.
77. Jansson D, Rustenhoven J, Feng S, Hurley D, Oldfield R L, Bergin P S, et al. A role for human brain pericytes in neuroinflammation. Journal of neuroinflammation. 2014; 11:104. doi: 10.1186/1742-2094-11-104.

1-13. (canceled)

14. A method of improving motor deficits in a mammal having a neurodegenerative condition and suffering motor deficits comprising administering an effective amount of (E)-4-(2-nitrovinyl) benzoic acid or a pharmaceutically acceptable salt thereof to the mammal thereby to improve the motor deficits.

15. The method according to claim 14, wherein the mammal has at least one neurodegenerative condition selected from the group consisting of Alzheimer's Disease, Parkinson's Disease, multiple sclerosis, Huntington's Disease, amyotrophic lateral sclerosis (ALS), spinal muscular atrophy, muscular dystrophies prion-related diseases, cerebellar ataxia, Friedrich's ataxia, SCA, Wilson's disease, RP, Gullian Barre syndrome, Adrenoleukodystrophy, Menke's syndrome, cerebral autosomal dominant arteriopathy with subcortical infarcts (CADASIL), Charcot Marie Tooth diseases, neurofibromatosis, von-Hippel Lindau, Fragile X, spastic paraplegia, tuberous sclerosis complex, Wardenburg syndrome, spinal motor atrophies, Tay-Sach's, Sandoff disease, familial spastic paraplegia, myelopathies, radiculopathies, encephalopathies associated with trauma, radiation, drugs and infection, and disorders of the sympathetic nervous system (e.g., Shy Drager (familial dysautonomia), diabetic neuropathy, drug-induced and alcoholic neuropathy), and combinations thereof.

16. The method according to claim 14, wherein the neurodegenerative condition is amyotrophic lateral sclerosis (ALS).

17. The method according to claim 14, wherein the neurodegenerative condition is Parkinson's disease.

18. The method according to claim 14, wherein the neurodegenerative condition is Alzheimer's disease.

19. The method according to claim 14, wherein (E)-4-(2-nitrovinyl) benzoic acid is administered.

20. The method according to claim 14, wherein the mammal is a human subject.

21. A method of reducing motor neuron degeneration in a mammal having amyotrophic lateral sclerosis (ALS) and suffering motor neuron degeneration comprising administering an effective amount of (E)-4-(2-nitrovinyl) benzoic acid or a pharmaceutically acceptable salt thereof to the mammal thereby to reduce motor neuron degeneration.

22. The method according to claim 21, wherein (E)-4-(2-nitrovinyl) benzoic acid is administered.

23. The method according to claim 21, wherein the mammal is a human subject.

24. A method of improving motor deficits in a mammal having a neurodegenerative condition comprising (i) diagnosing motor deficits in a mammal having a neurodegenerative condition and (ii) administering to said mammal having a diagnosis of motor deficits an effective amount of (E)-4-(2-nitrovinyl) benzoic acid or a pharmaceutically acceptable salt thereof to the mammal thereby to improve the motor deficits.

25. The method according to claim 24, wherein the neurodegenerative condition is amyotrophic lateral sclerosis (ALS).

26. The method according to claim 24, wherein the neurodegenerative condition is Parkinson's disease.

27. The method according to claim 24, wherein the neurodegenerative condition is Alzheimer's disease.

28. The method according to claim 24, wherein (E)-4-(2-nitrovinyl) benzoic acid is administered.

29. The method according to claim 24, wherein the mammal is a human subject.
